(19) United States
(12) Patent Application Publication
(10) Pub. No.: US 2023/0390366 A1
(43) Pub. Date: Dec. 7, 2023

(54) PARAPHARMACEUTICAL OR PHARMACEUTICAL COMPOSITION ADMINISTRABLE TO A LIVING BEING, PREFERABLY A HUMAN BEING, COMPRISING AT LEAST ONE ENZYME FOR THE TREATMENT AND/OR PREVENTION OF BACTERIAL INFECTIONS INVOLVING BIOFILM FORMATION

(71) Applicants: ONELIFE S.A., Louvain-la-Neuve (BE); UNIVERSITE CATHOLIQUE DE LOUVAIN, Ottignies-Louvain-la-Neuve (BE)

(72) Inventors: Laetitia FONTAINE, Corroy-le-Grand (BE); Aline LARDINOIS, Kraainem (BE); Houssein CHALHOUB, Woluwé-Saint-Lambert (BE); Françoise VAN BAMBEKE, Bruxelles (BE); Albert RUIZ-SORRIBAS, Bruxelles (BE)

(21) Appl. No.: 18/249,182

(22) PCT Filed: Oct. 18, 2021
(86) PCT No.: PCT/EP2021/078824

§ 371 Date: Apr. 14, 2023

(30) Foreign Application Priority Data
Oct. 16, 2020 (BE) 2020/5726

(51) Int. Cl. A61K 38/46; A61K 8/65; A61K 38/47; A61K 38/44; A61K 38/48; A61K 45/06; A01N 63/50; A01P 1/00; A61Q 3/00; A61Q 11/00; A61P 31/04

(52) U.S. Cl. CPC A61K 38/465; A61K 8/65; A61K 38/47; A61K 38/44; A61K 38/48; A61K 45/06; A01N 63/50; A01P 1/00; A61Q 3/00; A61Q 11/00; A61K 38/482; A61P 31/04

(57) ABSTRACT

A parapharmaceutical or pharmaceutical composition is administrable to living beings, and in particular to human beings. The composition includes at least one endoribonuclease enzyme chosen from the EC 3.1.30 and EC 3.1.31 classes, preferably in a content of from 10 to 1000 U/ml, preferably 50 to 500 U/ml, preferably from 100 to 500 U/ml. The composition is for curative or preventive treatment for dermatological infections or for infections which develop on superficial or deep burns and wounds.

The present invention relates to a parapharmaceutical or pharmaceutical composition which can be administered to living beings and in particular to humans, comprising at least one enzyme and to its use as a potentiator in the treatment and/or prevention of bacterial infections involving biofilm formation, such as post-implantation infections, related to in situ medical implants, and therefore implanted implants, and various infections of the body such as acute or chronic infections of wounds, burns, infections of the oral cavity, digestive tract, urinary system, respiratory system.

A biofilm is a viscous film that develops on all surfaces, following the adhesion of microorganisms to such surfaces and the secretion by these of polymers that cover them and facilitate their adhesion. Biofilms thus form a protective layer around microorganisms and represent a recurring source of contamination of the surrounding environment which poses major problems in terms of health, for example in hospital environments.

More specifically, the accumulation of polymers secreted by bacteria creates a matrix composed essentially of polysaccharides, DNA, proteins as well as lipids which protects these microorganisms from external aggressions and which is highly resistant to conventional cleaning and disinfection procedures. Microorganisms therefore develop easily within this protective matrix and contaminate the surrounding environment by forming a particularly critical reservoir that is difficult to eliminate.

It is recognised that there are two issues caused by the presence of biofilms. First, as indicated above, they represent a source of permanent contamination and are very difficult to remove by conventional methods, even when using the most aggressive methods. Indeed, conventional disinfectants are very often ineffective because it can be seen that they fail to reach the microorganisms which are protected by the biofilm matrix composed of polysaccharides, DNA, proteins and lipids.

Second, a biofilm is mixed, in the sense that it is initially developed by certain bacterial strains but may harbour others, with these strains living and growing in colonies. However, these colonies promote communication between bacteria and, among other things, the exchange and propagation of resistance genes carried by certain bacteria. Biofilms resulting from these gene exchanges become even more difficult to eliminate and it is necessary to resort to increasingly powerful means of disinfection or treatment which, still, frequently face major problems in terms of resistance and/or tolerance.

The protective matrix of the bacteria forming the biofilms is so resistant that it forms a strong barrier protecting the bacteria from microbicidal agents (antibiotics and/or biocides) which could fight the microorganisms and therefore the infections linked to the presence of biofilms, including infections of the human body associated with the presence of biofilm. Currently, conventional treatments based on different antibiotics and/or different biocides (disinfectants, etc.), even when they are, in certain cases, combined with other compounds (formulated detergents and/or sequestrants and/or dispersants and/or surfactants), do not act effectively enough because they do not penetrate the biofilm or only penetrate the biofilm's thickness to a certain extent. Furthermore, microbicides may be inhibited by certain molecules forming this matrix. As a result, current treatments are only partially effective, and act only on the surface of the biofilm, since the biofilm matrix effectively protects the bacteria from dehydration phenomena, from the action of antibiotics and biocides (and more generally from microbicidal molecules), from phagocytosis and acids. In this sense, it is also generally accepted that biofilms display up to 1000 times more resistance to microbicides compared with planktonic bacteria (not protected by a biofilm).

It is therefore understood that the treatment and/or prevention of bacterial infections involving the formation of biofilms is a serious challenge because bacteria are protected from microbicidal agents and external aggressions.

In a hospital, dental or veterinary environment, the situation is extremely critical since many microorganisms responsible for the formation of biofilms are found in numerous places, both in individual patients/animals (wounds, sores, burns, respiratory system, etc.) and in the environment (operating room, dental practices and equipment, surgical equipment, maintenance equipment for this equipment, endoscopes, urinary catheters, catheters, dental implants or prostheses, medical equipment, dialysis or assisted ventilation equipment for individuals, etc.) and surfaces (floors, walls, operating tables, etc.).

At the same time, wound management, a rapidly evolving field, is also significantly affected by bacterial infections involving the formation of biofilms. Found in more than 90% of chronic wounds, biofilm is a major obstacle to healing, since it increases the inflammatory response and delays the wound-healing process.

Acute or chronic wounds/injuries include surgical wounds, burns, venous and arterial leg ulcers, diabetic foot ulcers, pressure ulcers (bedsores), burns, etc. Chronic ulcers are extremely painful and difficult to treat. A wound is said to be chronic when no sign of healing can be seen after 4 to 6 weeks.

Wound management consists of two segments: the traditional wound management segment and the advanced active wound management segment. Traditional wound care is all about dressing in a dry environment to cover wounds and protect them from the environment.

Advanced active management of wounds/injuries covers any treatment that provides strong support for the cicatrisation and healing of wounds, sometimes including the debridement of damaged tissue. This treatment will stimulate the growth of new tissues and control infection and pain.

The field of personal hygiene or cosmetic care is also affected by bacterial infections linked to the formation of biofilms. Above we mentioned dental practices with regard to post-implantation infections following the fitting of dental prostheses (peri-implantitis). However, there are other applications where biofilms have a negative impact on health. For example, we know that teeth, gums, and dental prostheses are excellent supports for bacteria as a result of the humid oral environment which is rich in microorganisms, where they are located. It is reported that biofilm forming dental plaque is a major risk factor for complications such as cavities, periodontal disease and gingivitis and it is recognised that when biofilm becomes thick, it shows resistance to chemicals (antibiotics, disinfectants). In addition, respiratory disease is often linked to periodontal disease thanks to the transfer of microorganisms from the mouth to the respiratory tract. However, it is possible to reduce the health risks associated with dental plaque by preventing and/or reducing its formation through mechanical action.

In this regard, Kaplan et al. describe a mutated dispersin B which is a β-N-acetylglucosaminidase (of class EC 3.2.1.52) in oral hygiene products such as toothpaste or mouthwash. Another example is in the treatment of onychomycosis (fungal nail infection) or ocular lenses where biofilms are also involved or even acne. For example, WO 2020/0185685 describes a composition for the treatment of acne, which may contain “an RNA inhibitor”.

From the above, it is clear that biofilms pose a serious challenge, particularly in the care sector, in particular health care (hospitals, dental practices, etc.) and veterinary care and even personal hygiene.

This problem is made all the more critical by the fact that biofilms involve bacteria responsible for potentially fatal infections in individuals, for example in individuals developing an infection caused by bacteria of the Staphylococci or Enterobacteriaceae genus which prove to be multi-resistant to new-generation antibiotics. It is therefore necessary to take every possible precaution to prevent the formation and development of biofilms.

Compositions comprising at least one enzyme for dispersing a biofilm are well known in the prior art. For example, documents US2009/0130082 and WO2009/121183 relate to compositions comprising a bovine DNase I (endodeoxyribonuclease I of class EC 3.1.21) to disperse the biofilm and an antibiotic (microbicidal agent) to kill the bacteria released. According to these prior documents, these compositions are used in particular during the manufacture/preparation of medical devices for treating wounds. To this end, medical devices are covered (coated) or impregnated with a composition comprising a DNase I and an antibiotic. More specifically, these prior documents essentially teach that such compositions are used to prepare medical devices and to disinfect the skin or the surrounding environment before a medical device is inserted or implanted. In this regard, the compositions disclosed in these prior documents are essentially used as coatings or as pre-procedure rinsing solutions before surgery, for example.

U.S. Pat. No. 10,328,129 describes the use of nucleases for the treatment of eye conditions.

WO 01/93875 describes a composition for the treatment of a biofilm comprising a first enzyme component provided with an anchor for the degradation of biofilm structures and a second enzyme component provided with an anchor having a direct bactericidal effect. DNAses are indicated as belonging to this second enzyme category.

US 2017/355930 describes a detergent product comprising a nuclease and an amine.

With this in mind, application EP 3468583 is known, which describes a composition comprising at least one enzyme such as for example a DNase I and at least one microbicidal molecule for the prevention or treatment of post-implantation biofilm infections.

Furthermore, the issue of biofilm infections is not limited to post-implantation biofilm infections. Indeed, biofilms are also involved in chronic infections in the bodies of animals. As explained above, these infections can be found, for example, in wounds/sores present on the surface of the human body or the body of mammals.

Patent application WO2006/133523 is also known, which describes a composition comprising at least one enzyme used in the treatment of skin problems and wounds. This composition includes at least one peroxidase for the purpose of generating antimicrobial hypothiocyanite radicals from H₂O₂ and thiocyanate for the treatment of infections.

Unfortunately, as can be seen, the prior art contains many teachings given the diversity of infections, such as those involving biofilms. In some cases, the authors of research work have sought to identify an enzyme that will have a specific and/or targeted action against biofilms, while others have focused on an antimicrobial action without taking into account the biofilm matrix.

Alternatively, we know commercial solutions such as EnziQure® which is a multi-enzymatic detergent used for the curative dissolution of biofilm and the deep cleaning of surgical instruments and endoscopes. EnziQure® is a formulated product that contains a non-ionic surfactant base, dispersing and sequestering agents and an enzyme cocktail containing 7 enzymes to be able to degrade the biofilm matrix of many bacterial species (its anti-biofilm action has been demonstrated on more than 15 strains), with enzymes being found in high concentrations to ensure maximum effectiveness.

Although very effective for cleaning medical instruments, this solution is a Class I medical device which cannot be administered to living beings, in particular to humans due to its detergent and enzymatic composition which does not have the pharmaceutical grade required for its use as an active substance (medicament).

There is therefore currently a need to develop alternative formulations which can be applied to the body of a living being, in particular to the human or animal body, in particular for the treatment and/or prevention of bacterial infections involving biofilm formation and having an efficacy similar to that of the EnziQure® product of the applicant of the present patent application.

The aim of the present invention is therefore to provide an enzyme composition which can be administered to humans both externally and internally and the efficacy of which is similar to that of the detergent product EnziQure® with respect to the dissolution of the biofilm matrix and offering multi-purpose use.

The terms “multi-purpose use” or “multiple use” refer to use on a wide range of strains of bacteria and for a range of applications, namely a use that has a broad spectrum of actions.

Indeed, in order for it to be possible to industrially exploit a formulation for the treatment and/or prevention of bacterial infections involving biofilm formation, it is advantageous to have a formulation that has a broad spectrum of action, i.e. capable of dissociating different types of biofilm associated with different types of infection. Indeed, the composition of a biofilm varies according to the bacteria that form it, and therefore the location and type of infection. Such a formulation would therefore be suitable for various applications, and would avoid the need to create different formulations depending on the type of infection targeted, because this approach involves logistical complications hindering the industrial exploitation of such a formulation. The possibility of obtaining a formulation which is active in the treatment and/or prevention of bacterial infections involving the formation of biofilms resulting from a wide range of bacterial strains and intended for different applications allows at least the manufacture of a concentrate which can then be diversified for certain applications or to formulate a multi-purpose composition.

To solve this problem, the invention provides for the use of a parapharmaceutical or pharmaceutical composition which can be administered to living beings, in particular to human beings, comprising at least one endoribonuclease enzyme selected from the group consisting of enzymes belonging to classes EC 3.1.30 (for example EC.3.1.30.1 or EC.3.1.30.2) and EC 3.1.31 and mixtures thereof, to potentiate a microbicidal agent, in particular an antibiotic, a phage, an antifungal, or a disinfectant, in the treatment and/or prevention of bacterial infections involving biofilm formation.

By way of example, such an enzyme is Denarase® or Benzonase® endonuclease. They are preferably present at a concentration of 10 to 1000 U/ml, preferably 50 to 500 U/ml, preferably 100 to 500 U/ml.

Concentrations according to the present invention of said at least one endoribonuclease enzyme selected from the group consisting of enzymes belonging to classes EC 3.1.30 (EC.3.1.30.1 or EC.3.1.30.2) and EC 3.1.31 are for example 200 U/ml to 650 U/ml, more particularly 250 U/ml to 550 U/ml, preferably 300 U/ml, or even 350 U/ml to 500 U/ml. When several endoribonucleases are used together, either each present at the above concentration, or together, they make up the above concentration.

Concentration is expressed according to the present invention in terms of U/ml. One unit (U) is equal to the amount of protein that causes a change in absorbance at 260 nm of 1.0 in 30 minutes at 37° C.

As can be seen, the composition used according to the present invention is a parapharmaceutical or pharmaceutical composition, which can be administered to living beings, in particular to humans. When the composition is pharmaceutical, it is essentially endotoxin-free.

The term “substantially endotoxin-free composition” refers to a composition which contains no or very little toxic heat-stable lipopolysaccharide material. Typically depending on the organism from which the enzyme originates, the enzyme solution often contains endotoxins, which are present in the outer membrane of Gram-negative bacteria. Endotoxins are released during the extraction of the enzyme from the cell during cell lysis. Such enzymes are widely marketed, but are by no means suitable for pharmaceutical use, especially since they are generally not produced according to Good Manufacturing Practices. An endotoxin-free enzyme can be obtained either by production in gram+ bacteria, or in other hosts that do not contain endotoxins in their membrane, or by purification.

Said at least one endoribonuclease enzyme of the composition used according to the present invention displays activity on RNA and on DNA. Its enzymatic activity hydrolyses DNA and RNA substrates into 5′-phospho(mono/oligo)nucleotide products (EC 3.1.30: e.g. EC.3.1.30.1 or EC.3.1.30.2) and/or 3′-phospho(mono/oligo)nucleotide products (EC 3.1.31).

As indicated above, the compositions currently known and developed are compositions comprising, for the most part, an endodeoxyribonuclease (Dnase 1) of class EC 3.1.21 for hydrolysing DNA and/or a polysaccharide hydrolase (Dispersin B) to hydrolyse the polysaccharides present in the biofilm matrix.

The enzyme pharmaceutical composition used according to the present invention meets very strict manufacturing conditions. The composition according to the present invention is manufactured in compliance with good manufacturing practices (cGMP) which are directives that have become obligatory in the pharmaceutical field, both with regard to the production chain of a medicament and with regard to handling in the laboratory. These guidelines require production tracking and quality control to ensure the efficacy, purity and reproducible safety of a product for pharmaceutical use. In particular, the enzyme composition according to the invention meets the pharmaceutical specifications in terms of harmlessness, quantities of endotoxin and microorganisms (virus, Mycoplasma, bacteria) present, purity and absence of products of animal origin or antibiotics used in its manufacture. These specifications constitute the prerequisites for therapeutic use in the human body, whether for external or internal use.

It has in fact been identified according to the present invention that it was possible to produce such an enzyme in a Gram+ bacterial strain (or via any other fermentation process not involving Gram− bacteria), which does not produce endotoxin and the production and purification of which complies with good manufacturing practices. Other production methods are of course possible, including Gram− bacteria, subject to an appropriate downstream purification process. For example, benzonase endonuclease also complies with good manufacturing practices.

The parapharmaceutical composition comprising the nuclease (endoribonuclease: RNase) selected from classes EC 3.1.30 (EC.3.1.30.1 or EC.3.1.30.2) or EC 3.1.31 does not specifically need to be of pharmaceutical grade. By extension, such a parapharmaceutical composition can be a medical device such as a dressing or a toothpaste, and can therefore be a personal hygiene product, such as a lotion or a toothpaste.

In addition, and surprisingly, the use of the composition according to the invention allows a reduction in the biomass of biofilms originating from numerous bacterial strains (and even yeasts when subtilisin and/or glucanase are added) and thus allows the microbicidal agent to access the microorganisms present inside the biofilm for the treatment of the biofilm infection and thus potentiates its effect. The use of the composition according to the present invention creates a synergistic effect in the enzyme composition and the microbicidal agent used.

Indeed, while other RNAses belonging to the EC4.6.1 classification, such as RNase I and RNase A (although existing on the market at pharmaceutical grade and therefore administrable to the body of a living being, in particular a mammal, more particularly a human being) have no effect on biofilm infections or a limited effect on certain types of biofilms only, it has surprisingly been demonstrated that an endoribonuclease selected specifically from the class EC 3.1.30 (EC.3.1.30.1 or EC.3.1.30.2) or EC 3.1.31 makes it possible to drastically reduce the biomass of biofilms and thus to potentiate the microbial agent which, without this composition, is not capable of entering the biofilm and treating the infection, and this is for biofilms of various strains of bacteria and with a non-specific use of the biofilm infection to be treated or already treated.

The non-specific endoribonuclease activity of enzymes belonging to classes EC 3.1.30 (EC.3.1.30.1 or EC.3.1.30.2) or EC 3.1.31 makes it possible to digest DNA and RNA. Contrary to all expectations, these enzymes already lead to a drastic reduction in the biomass of biofilms when they are used alone in a formulation according to the present invention, and the drastic reduction in the biomass of biofilms is similar to that observed for the multi-enzyme commercial formulation EnziQure®. In addition, according to the present invention, an efficacy against several types of biofilms is already achieved with a single enzyme, which is particularly advantageous in a parapharmaceutical or pharmaceutical composition where the least possible ingredients are sought to facilitate marketing authorisations.

The dependent claims relate to other advantageous forms of use. According to a first use of a composition according to the present invention, the treatment and/or prevention of bacterial infections involving biofilm formation is a curative or preventive treatment for dermatological infections or for infections caused by superficial or deep wounds. These dermatological bacterial infections or those caused by superficial or deep wounds are, for example, wounds or burns. These infections can develop on chronic or acute wounds, burns, at the site where a medical device is inserted. The elimination or reduction of biofilm speeds up the healing process and healing of wounds and burns.

According to a second use of a composition according to the present invention, the treatment and/or prevention of bacterial infections involving biofilm formation is a curative or preventive treatment for post-implantation infections associated with the infection caused by a medical device implanted in the body or of tissues around a medical device implanted in the body. The use of a composition according to the present invention will allow in situ treatment of the biofilm formed on an infected implanted medical device or on the tissues around said implanted medical device. For example, an infected implanted prosthesis should not be taken out of the human body to be cleaned. The infected internal tissues and the prosthesis can be cleared via in situ administration.

According to a third use according to the invention, the parapharmaceutical or pharmaceutical composition which can be administered to living beings, in particular to human beings in the treatment and/or prevention of bacterial infections involving biofilm formation is used for the manufacture of a treatment or in a personal hygiene or cosmetic treatment, such as for example nail care, an oral solution, a mouthwash, a toothpaste, an eye bath, an eye lens cleaning solution, a cleaning solution for braces, dentures, toothbrushes, skin care for acne.

According to another use according to the invention, the parapharmaceutical or pharmaceutical composition is used to clean accessories that come into contact with the human body, for example piercings.

Advantageously, the composition further comprises a series of additional enzymes, for example one, two, three, four, five, six, seven, eight enzyme(s).

More particularly, an enzyme from said series of additional enzymes is selected from the group consisting of glycosidases (EC 3.2.1), deoxyribonucleases (EC 3.1.21), oxidoreductases (EC 1.), carboxylic ester hydrolases (EC 3.1.1), proteases and peptidases (EC 3.4.), and mixtures thereof.

The class of glycosidases includes polysaccharide hydrolases such as cellulase (EC 3.2.1.4), glucanase (e.g. endo-β-1 3-glucanase, such as for example EC.3.2.1.39, CAS 9025-37-0, CAS 37340-57-1, especially CAS 144941-36-6, which is endo-β-1 3-glucanase from Bacillus subtilis: endoglucanase is also called “Lyticase”), dispersin B (EC 3.2.1.52), hexosaminidase (EC 3.2.1.52), alpha-amylase (EC 3.2.1.1), mannanase (EC 3.2.1.78 and EC 3.2.1.101), hyaluronidase (EC 3.2.1.35), glucosidase (EC 3.2.1.21 and EC 3.2.1.20), galactosidase (EC 3.2.1.22 and EC 3.2.1.23), chitinase (EC 3.2.1.14), chitosanase (EC 3.2.1.132), fructanase (EC 3.2.1.80), dextranase (EC 3.2.1.11), lysozyme (EC 3.2.1.17) and pectinase (EC 3.2 0.1.15). This class also includes polysaccharide lyases such as alginate lyase (EC 4.2.99.4 and EC 4.2.2.26) and hyaluronate lyase (EC 4.2.2.1).

The class of oxidoreductases includes for example laccase (EC 1.10.3.2), peroxidases (EC 1.11.1) lactoperoxidase (EC 1.11.1.7), haloperoxidase (EC 1.11.2.1), myeloperoxidase (EC 1.11.2.2) and glucose oxidase (EC 1.1.3.4).

The class of carboxylic ester hydrolases (EC 3.1.1) includes for example lipase (EC 3.1.1.3).

The class of proteases and peptidases (EC 3.4.) includes for example flavourzyme or proteases such as Blaze®Pro, subtilisin (EC 3.4.21.62), bromelain (EC 3.4.22.33), collagenase (EC 3.4.24.3), papain (EC 3.4.22.2), trypsin (EC 3.4.21.4) and proteinase K (EC 3.4.21.64). Subtilisin is preferred.

A related aspect of the present invention is moreover the use of subtilisin for the (parapharmaceutical) treatment of a biofilm comprising Candida sp., such as Candida albicans. Subtilisin can be applied alone, or in combination with the other enzymes above, either used sequentially (subtilisin then one or more other enzymatic activities or one or more enzymatic activities, followed by subtilisin), or combined.

Conversely, a related aspect of the present invention is subtilisin for the treatment of a biofilm comprising Candida sp., such as Candida albicans in a patient. Subtilisin can be administered alone, or in combination with the other enzymes above, either used sequentially (subtilisin then one or more other enzymatic activities or one or more enzymatic activities, followed by subtilisin), or combined.

Thus, a related aspect of the present invention is a method of treating a biofilm comprising Candida sp., such as Candida albicans, with the method comprising the following steps:
the biofilm is reacted with subtilisin:
an antifungal is applied (administered).
   This method advantageously comprises the following additional steps of applying one or more other enzymatic activities to the biofilm and/or of applying (administering) an anti-infective, for example an antibiotic.

In a preferred embodiment of the use of a composition according to the present invention comprising enzymes belonging to classes EC 3.1.30 (EC.3.1.30.1 or EC.3.1.30.2) and/or EC 3.1.31, or subtilisin, the composition comprises at least one second enzyme selected from the group of glycosidases (EC 3.2.1).

In another preferred embodiment of the use of a composition according to the present invention comprising enzymes belonging to classes EC 3.1.30 (EC.3.1.30.1 or EC.3.1.30.2) or subtilisin, the composition comprises at least a third enzyme selected from the group consisting of glycosidases (EC 3.2.1), deoxyribonucleases (EC 3.1.21), oxidoreductases (EC 1), carboxylic ester hydrolases (EC 3.1.1), peptidases and proteases (EC 3.4), and mixtures thereof, in particular selected from the group consisting of glycosidases (EC 3.2.1) and peptidases (EC 3.4) (the addition of a peptidase is not preferred when the enzyme according to the invention is subtilisin).

In another preferred embodiment of the use of a composition according to the present invention comprising enzymes belonging to classes EC 3.1.30 (EC.3.1.30.1 or EC.3.1.30.2) or subtilisin, the composition comprises at least a fourth enzyme selected from the group consisting of glycosidases (EC 3.2.1), deoxyribonucleases (EC 3.1.21), oxidoreductases (EC 1), carboxylic ester hydrolases (EC 3.1.1), proteases and peptidases (EC 3.4) (the addition of a protease or a peptidase is not preferred when the enzyme according to the invention is subtilisin) for the manufacture of a medicament for the treatment and/or prevention of post-implantation diseases associated with the infection of a medical device implanted in the body.

In an embodiment of the use of a composition according to the present invention, the medical device is an orthopaedic implant.

In a particular use of the composition according to the present invention, the treatment and/or prevention of bacterial infections involving biofilm formation is a curative or preventive treatment for a bacterial infection of tissues surrounding an implanted orthopaedic implant or of the implanted orthopaedic implant.

When subtilisin and/or lyticase (endo-β-1 3-glucanase) is present in the composition according to the invention, or added, the composition according to the invention is (also) used for the treatment and/or the prevention of infection by Candida sp. (Candida albicans) involved in the biofilm, including mixed biofilms comprising Candida and one or more bacterial species, which are the most difficult biofilms to treat.

In another particular use of the composition according to the present invention, the treatment and/or prevention of bacterial infections involving biofilm formation is a curative or preventive treatment for a bacterial infection of tissues surrounding an implanted dental implant or of the implanted dental implant.

In yet another particular use of the composition according to the present invention (comprising enzymes belonging to classes EC 3.1.30 (EC.3.1.30.1 or EC.3.1.30.2), the treatment and/or prevention of bacterial infections involving biofilm formation is a curative or preventive treatment for a bacterial infection of tissues surrounding a catheter, a cannula, a probe, a prosthesis, a endosseous implant, a zygomatic implant, an orthodontic implant, a dental prosthesis, a retainer, a valve, a drain, a stent, a tube for artificial respiration or an implanted screw or of a catheter, a cannula, a probe, a prosthesis, an endosseous implant, a zygomatic implant, an orthodontic implant, a dental prosthesis, a retainer, a valve, a drain, a stent, a tube for artificial respiration or an implanted screw.

Alternatively for the composition according to the present invention comprising subtilisin, the treatment and/or the prevention of infections comprising Candida Sp. (Candida albicans) involving biofilm formation is a curative or preventive treatment for a fungal infection (potentially in addition to bacterial infection)
of tissues surrounding a catheter, a cannula, a probe, a prosthesis, an endosseous implant, a zygomatic implant, an orthodontic implant, a dental prosthesis, a retainer, a valve, a drain, a stent, a tube for artificial respiration or an implanted screw or of
a catheter, a cannula, a probe, a prosthesis, an endosseous implant, a zygomatic implant, an orthodontic implant, a dental prosthesis, a retainer, a valve, a drain, a stent, a tube for artificial respiration or an implanted screw.

In an advantageous use of the composition according to the present invention (comprising enzymes belonging to classes EC 3.1.30 (EC.3.1.30.1 or EC.3.1.30.2), the treatment and/or the prevention of bacterial infections involving biofilm formation comprises an application of said composition on a wound, an infection, tissues or a medical device, preferably an application of a woven or non-woven support impregnated with said parapharmaceutical or pharmaceutical composition.

The support can be a foam, a sponge, a film, a hydrocolloid dressing, a dressing based on alginate, polyurethane, a hydrogel, a gauze pad, a bandage, a wipe, a dressing, etc.

In another advantageous use of the composition according to the present invention (comprising enzymes belonging to classes EC 3.1.30 (EC.3.1.30.1 or EC.3.1.30.2) and/or subtilisin), the treatment and/or prevention of bacterial infections involving biofilm formation comprises an application of said composition to a wound, an infection, tissues, preferably an application of a dressing in the form of a gel comprising said parapharmaceutical or pharmaceutical composition.

In another advantageous use of the composition according to the present invention (comprising enzymes belonging to classes EC 3.1.30 (EC.3.1.30.1 or EC.3.1.30.2)), the treatment and/or prevention of bacterial infections involving biofilm formation comprises an application of said composition to a wound, infection, tissues or a medical device, preferably an application of an aqueous, preferably buffered solution of said parapharmaceutical or pharmaceutical composition, by infiltration, by irrigation, by injection, by percutaneous application, by inhalation, by mouthwash or eyewash, by dabbing.

The aqueous solution of said parapharmaceutical or pharmaceutical composition according to the present invention is for example in concentrated form or in diluted and/or ready-to-use form.

In yet another advantageous use of the composition according to the present invention (comprising enzymes belonging to classes EC 3.1.30 (EC.3.1.30.1 or EC.3.1.30.2) and/or subtilisin), the treatment and/or the prevention of bacterial infections involving biofilm formation comprises an application of said composition to a wound, infection, tissues or a medical device, preferably an application of a paste or a viscous solution comprising said parapharmaceutical composition or pharmaceutical, such as a toothpaste.

Advantageously, according to the present invention, said microbicidal agent, in particular said antibiotic, phage, antifungal or said disinfectant and said composition form a combination product for simultaneous, separate or staggered use over time.

Other embodiments of the use of a parapharmaceutical or pharmaceutical composition which can be administered to living beings, in particular to human beings, according to the present invention are indicated in the attached claims.

The present invention also relates to a parapharmaceutical or pharmaceutical composition which can be administered to living beings, in particular to human beings, comprising at least one endoribonuclease enzyme selected from classes EC 3.1.30, more particularly from class EC 3.1 0.30.2, and EC 3.1.31, preferably at a concentration of 10 to 1000 U/ml, preferably 50 to 500 U/ml, preferably 100 to 500 U/ml, such as for example 200 U/ml to 650 U/ml, more particularly 250 U/ml to 550 U/ml, preferably 300 U/ml, or even 350 U/ml to 500 U/ml and optionally one or more pharmaceutically acceptable excipients. The advantage of this is that the endoribonuclease enzyme displays activity on RNA and DNA and allows digestion of the biofilm matrix, which can thus potentiate the effect of a microbicidal agent while allowing a reduced number of enzymes to be used.

Advantageously, said at least one endoribonuclease enzyme of the parapharmaceutical or pharmaceutical composition according to the present invention is of bacterial origin. Particularly advantageously, the endoribonuclease enzyme comes from Serratia marcescens.

In a preferred embodiment, the composition according to the present invention further comprises a series of additional enzymes, for example one, two, three, four, five, six, seven, eight enzyme(s).

Advantageously, the composition according to the present invention comprises an enzyme from said series of additional enzymes of said composition which is selected from the group consisting of glycosidases (EC 3.2.1), deoxyribonucleases (EC 3.1.21), oxidoreductases (EC 1), carboxylic ester hydrolases (EC 3.1.1) and peptidases (EC 3.4), and mixtures thereof.

More particularly, according to the present invention, the composition comprises at least a second enzyme selected from the group of glycosidases (EC 3.2.1), for example dispersin B, a cellulase or an endoglucanase (endo-β-1 3-glucanase, lyticase).

In a preferred embodiment, the composition according to the present invention further comprises at least a third enzyme selected from the group comprising glycosidases (EC 3.2.1), deoxyribonucleases (EC 3.1.21), oxidoreductases (EC 1), carboxylic ester hydrolases (EC 3.1.1), peptidases and proteases (EC 3.4), for example dispersin B, a cellulase or a glucanase (endo-β-1 3-glucanase, lyticase) and a protease, such as subtilisin, or two enzymes selected from, for example, dispersin B, a cellulase and glucanase (endo-β-1 3-glucanase, lyticase).

In a preferred embodiment, the composition according to the present invention comprises at least a fourth enzyme selected from the group comprising glycosidases (EC 3.2.1), deoxyribonucleases (EC 3.1.21), oxidoreductases (EC 1), carboxylic ester hydrolases (EC 3.1.1); peptidases and proteases (EC 3.4), for example dispersin B, a cellulase and glucanase (endo-β-1 3-glucanase, lyticase), or a protease, such as subtilisin and two enzymes selected from dispersin B, a cellulase and glucanase (endo-β-1 3-glucanase, lyticase).

In a preferred embodiment, the composition according to the present invention comprises at least a fifth enzyme selected from the group comprising glycosidases (EC 3.2.1), deoxyribonucleases (EC 3.1.21), oxidoreductases (EC 1), carboxylic ester hydrolases (EC 3.1.1), peptidases and proteases (EC 3.4), for example a protease, such as subtilisin, and dispersin B, a cellulase and glucanase (endo-β-1 3-glucanase, lyticase).

In a preferred embodiment, the composition according to the present invention comprises at least a sixth enzyme selected from the group comprising gycosidases (EC 3.2.1), deoxyribonucleases (EC 3.1.21), oxidoreductases (EC 1), carboxylic ester hydrolases (EC 3.1.1), peptidases and proteases (EC 3.4).

In a preferred embodiment, the composition according to the present invention comprises at least a seventh enzyme selected from the group comprising gycosidases (EC 3.2.1), deoxyribonucleases (EC 3.1.21), oxidoreductases (EC 1), carboxylic ester hydrolases (EC 3.1.1), peptidases and proteases (EC 3.4).

In a preferred embodiment, the composition according to the present invention comprises at least a eighth enzyme selected from the group comprising gycosidases (EC 3.2.1), deoxyribonucleases (EC3.1.21), oxidoreductases (EC1), carboxylic ester hydrolases (EC3.1.1), peptidases and proteases (EC 3.4).

In a preferred embodiment, the composition according to the present invention further comprises at least one microbicidal agent such as an antibiotic, an antifungal, an antiseptic, one or more microbicidal peptides or phages, or an antibiotic and an antifungal, conditioned separately or with said endoribonuclease.

Preferably, the microbicidal molecule is selected from the group consisting of fluoroquinolones, glucopeptides, lipoglucopeptides, fusidic acid, penicillins, cephalosporins, carbapenems, monobactams, polymyxins, beta-lactams, macrolides, lincosamides, oxazolidinones, phenicols, tetracyclines, aminoglycosides, rifamycins, nitrofurans, sulfonamides, antifungal nitroimidazoles (echinocandins, fluorocytosines, azoles, griseofulvins, amphotericin), lytic enzymes (e.g. endolysins or lysozyme), N-acetylcysteine, quaternary ammonia, biguanides, amines, amidines, halogenated derivatives (including chlorhexidine in particular), microbicidal peptides, silver derivatives (Ag), H2O2, peracids, phenolic derivatives, aldehydes, alcohols, phages and mixtures thereof.

In another preferred embodiment, the composition according to the present invention further comprises a quorum sensing inhibitor.

One or more enzymes that inhibit “quorum sensing” within biofilms may still be provided as additional compounds. Quorum sensing is a means of communication between bacterial cells that allows them to coordinate action across the entire population. Quorum sensing regulates, among other things, the formation of biofilms (generally early stages), based on the secretion and diffusion of acyl homoserine lactone (in Gram− bacteria) or peptides (in Gram+ bacteria), which are specifically recognised by cells of the same population. In particular, mention may be made of acylase I and 2(5H)-furanone.

In a preferred embodiment, the composition according to the present invention further comprises an enzyme buffer selected from the group consisting of Tris-HCl, TGN, TBS, PBS, HEPES, MES, PIPES, MOPS, BES, TES, phosphate buffer and citrate buffer, containing or not containing 0 to 2 mM MgCl2, containing 0 to 2 mM CaCl2, and 0 to 500 mM NaCl.

In another preferred embodiment, the composition according to the present invention further comprises an enzyme buffer comprising 0 to 50% of stabilising agent (polyol, arginine, calcium formate, glucose).

In a preferred embodiment, the composition according to the present invention further comprises at least one surfactant.

In a preferred embodiment, the composition according to the present invention additionally comprises at least one preservative and/or one sequestrant and/or one dispersant.

In a preferred embodiment, the parapharmaceutical or pharmaceutical composition according to the present invention is in the form of a solution, for example a mouthwash, an eye bath, a lotion, an irrigation solution, a cleaning solution for dentures, toothbrushes.

In another preferred embodiment, the composition is a hydrophilic dressing, for example a hydrogel.

In a variant according to the present invention, the composition is in immobilised form on a woven or non-woven, dry support or on a medical device or in impregnated form on a woven or non-woven support.

In another advantageous variant of the present invention, the composition is in the form of a topical composition.

In yet another advantageous variant of the present invention, the composition is in the form of a sterile solution which can be administered by infiltration, by irrigation, by injection, by percutaneous application and by inhalation.

The present invention also relates to a parapharmaceutical or pharmaceutical composition, for use as a potentiator for a microbicidal agent, in particular an antibiotic, an antifungal, or a disinfectant, in the treatment and/or prevention of bacterial infections involving biofilm formation.

In a particular embodiment according to the present invention, the parapharmaceutical or pharmaceutical composition is intended for use in the treatment and/or prevention of bacterial infections involving biofilm formation is a treatment and/or prevention for acute or chronic dermatological infections or infections caused by superficial or deep wounds (burns and/or wounds).

In a particular embodiment according to the present invention, the parapharmaceutical or pharmaceutical composition is intended for use in the curative and/or preventive treatment of post-implantation infections associated with the infection of tissues around a medical device implanted in the body or of a medical device implanted in the body.

In an advantageous embodiment according to the present invention, the parapharmaceutical or pharmaceutical composition is intended for use in a personal hygiene or cosmetic treatment, such as for example nail care, an oral solution, a mouthwash, toothpaste, eye bath, eye lens cleaning solution, skin care for acne.

In another advantageous embodiment according to the present invention, the parapharmaceutical or pharmaceutical composition is intended for use in a curative or preventive treatment for a bacterial infection of tissues surrounding an implanted orthopaedic implant or of the implanted orthopaedic implant.

In another advantageous embodiment according to the present invention, the parapharmaceutical or pharmaceutical composition is intended for the curative or preventive treatment of a bacterial infection of tissues surrounding an implanted dental implant or of the implanted dental implant.

In another advantageous embodiment according to the present invention, the parapharmaceutical or pharmaceutical composition is intended for use in a curative or preventive treatment for a bacterial infection of
tissues surrounding a catheter, a cannula, a probe, a prosthesis, an endosseous implant, a zygomatic implant, an orthodontic implant, a dental prosthesis, a retainer, a valve, a drain, a stent, a tube for artificial respiration or an implanted screw or of
a catheter, a cannula, a probe, a prosthesis, an endosseous implant, a zygomatic implant, an orthodontic implant, a dental prosthesis, a retainer, a valve, a drain, a stent, a tube for artificial respiration or an implanted screw.

In yet another advantageous embodiment according to the present invention, the parapharmaceutical or pharmaceutical composition is intended for use in the treatment and/or prevention of bacterial infections involving biofilm formation which comprises an application of said composition on a wound, an infection, tissues or a medical device, preferably an application of a woven or non-woven support impregnated with said parapharmaceutical or pharmaceutical composition or on which said parapharmaceutical or pharmaceutical composition is immobilised, dry.

In yet another advantageous embodiment according to the present invention, the parapharmaceutical or pharmaceutical composition is intended for use in the treatment and/or prevention of bacterial infections involving biofilm formation which comprises an application of said composition on a wound, an infection, tissues, preferably an application of a dressing in gel form, more particularly a hydrogel comprising said parapharmaceutical or pharmaceutical composition.

In a variant according to the present invention, the parapharmaceutical or pharmaceutical composition is intended for use in the treatment and/or prevention of bacterial infections involving biofilm formation which comprises an application of said composition to a wound, an infection, tissues, preferably an application of a dressing in gel form, more particularly a hydrogel comprising said parapharmaceutical or pharmaceutical composition.

In a variant according to the present invention, the parapharmaceutical or pharmaceutical composition is intended for use in the treatment and/or prevention of bacterial infections involving biofilm formation which comprises an application of said composition to a wound, an infection, tissues or a medical device, preferably an application of an aqueous solution, preferably buffered, of said parapharmaceutical or pharmaceutical composition, by infiltration, by irrigation, by injection, by percutaneous application, by inhalation, by mouthwash or eye bath, by dabbing, by soaking.

In another variant according to the present invention, the parapharmaceutical or pharmaceutical composition is intended for use in the treatment and/or prevention of bacterial infections involving biofilm formation which comprises an application of said composition to a wound, an infection, tissues or a medical device, preferably an application of a paste or of a viscous solution comprising said parapharmaceutical or pharmaceutical composition, such as for example a toothpaste.

In yet another variant according to the present invention, the parapharmaceutical or pharmaceutical composition is intended for use as a combination product for simultaneous, separate or staggered use over time.

Finally, the composition according to the present invention is intended for surgical use.

Other embodiments of the parapharmaceutical or pharmaceutical composition according to the invention are indicated in the attached claims.

Other characteristics, details and advantages of the invention will be included in the description given below, on a non-limiting basis and with reference to the drawings and the examples.

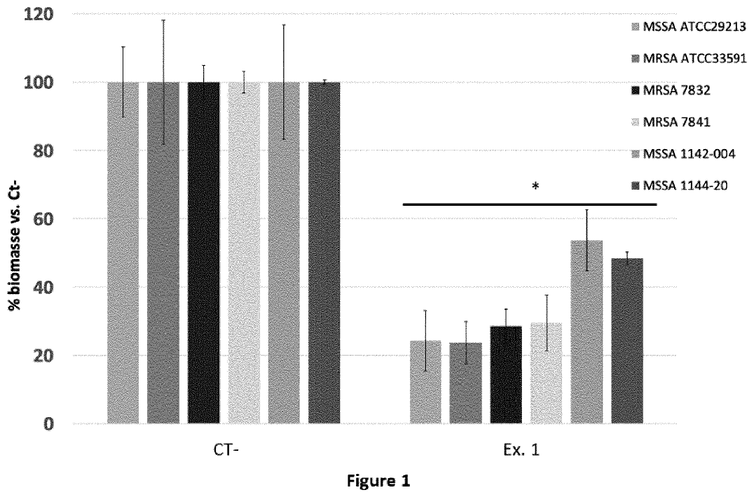

FIG. 1 is a graph which illustrates the reduction in the biomass percentage of the biofilm of several strains of Staphylococcus aureus, after contact with a composition according to the invention with a concentration of 500 U/ml for denarase.

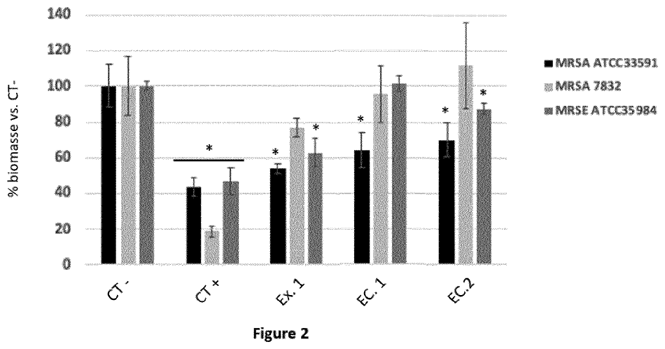

FIG. 2 is a graph which illustrates the reduction in the biomass percentage of the biofilm formed by different strains of S. aureus and Staphylococcus epidermidis, after contact with a composition according to the invention with a concentration of 100 U/ml for denarase compared with a composition comprising 100 U/ml of RNase I, with a composition comprising 100 U/ml for each enzyme in a mixture of RNase 1, RNase A and DNase 1.

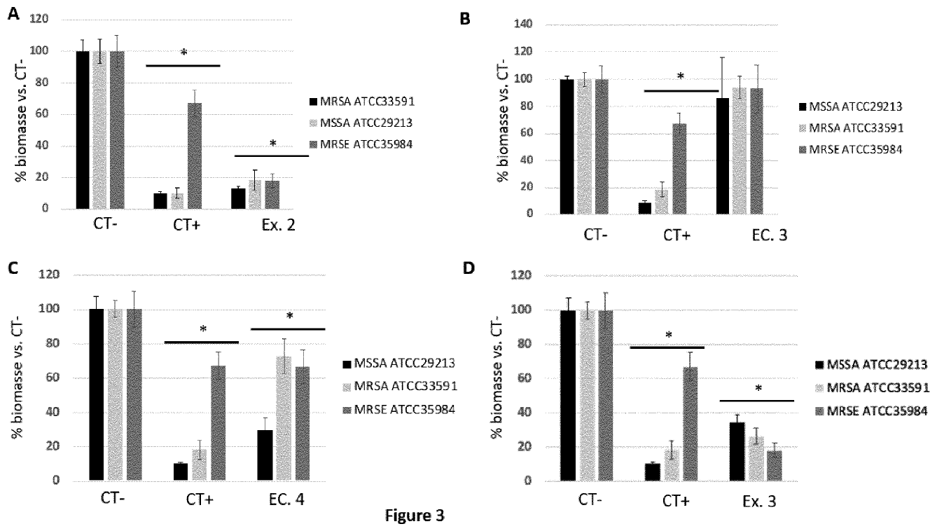

FIG. 3 shows a series of four graphs which illustrates the decrease in the biomass percentage of the biofilm of different strains of S. aureus and S. epidermidis, after contact with a composition according to the invention with a concentration of 500 U/ml for denarase compared with a composition comprising a CDD enzyme cocktail comprising 500 U/ml of denarase, 0.06 U/ml of dispersin B and 7 U/ml of cellulase, compared with a composition comprising 0.06 U/ml of dispersin B, compared with a composition comprising 7 U/ml of cellulase.

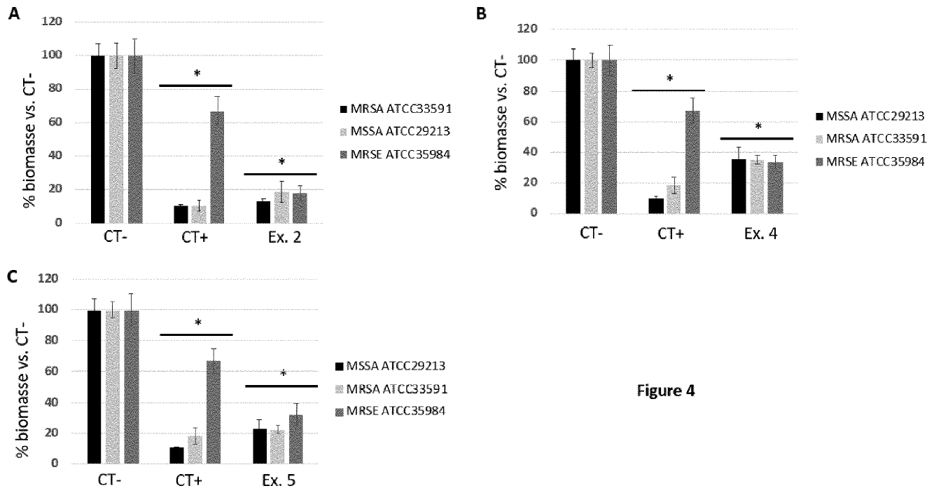

FIG. 4 shows a series of three graphs which illustrates the decrease in the biomass percentage of the biofilm of multiple strains of S. aureus and S. epidermidis, after contact with a composition comprising only 500 U/ml of denarase, compared with a composition comprising an enzyme cocktail comprising 500 U/ml of denarase and 0.06 U/ml of dispersin B, compared with a composition comprising an enzyme cocktail comprising 500 U/ml of denarase and 7 U/ml of cellulase.

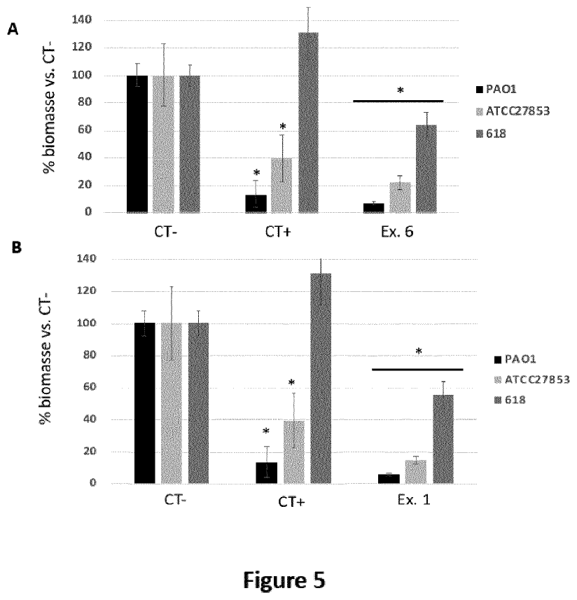

FIG. 5 shows a series of two graphs which illustrates the decrease in the biomass percentage of the biofilm of different strains of P. aeruginosa, after contact with a composition comprising 500 U/ml of denarase, compared with a composition comprising a CDD enzyme cocktail comprising 500 U/ml of denarase, 0.06 U/ml of dispersin B and 7 U/ml of cellulase.

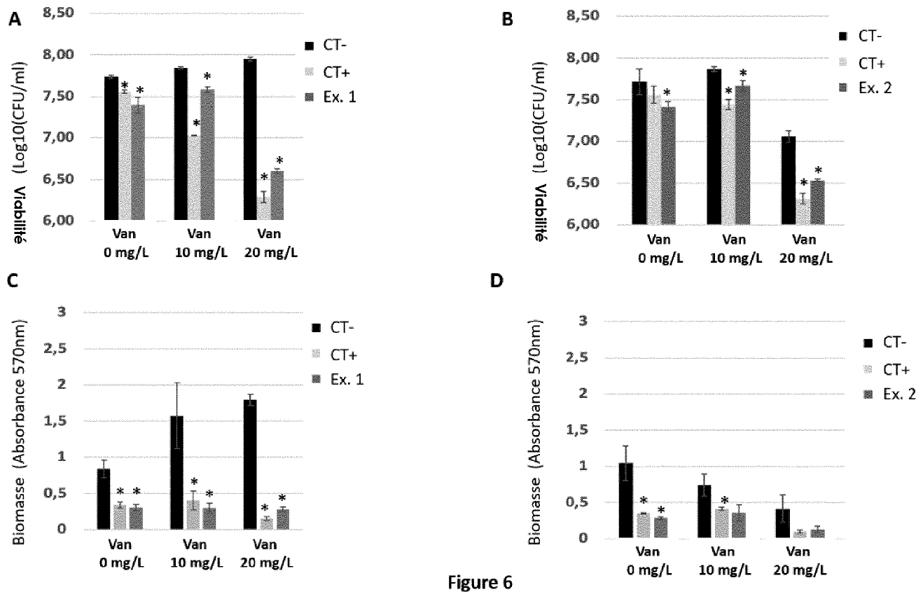

FIG. 6 shows a series of four graphs which illustrates the reduction in viability (expressed in log₁₀ (CFU/ml) of the S. aureus ATCC33591 biofilm after curative treatment of the biofilm with a composition according to the invention comprising denarase at 500 U/ml followed by 24 hours of incubation with different vancomycin concentrations of 0, 10, and 20 mg/L, as well as the reduction in the biomass (expressed in absorbance at 570 nm) of the S. aureus ATCC33591 biofilm after curative treatment of the biofilm with a composition according to the invention comprising denarase at 500 U/ml followed by 24 hours of incubation with different vancomycin concentrations of 0, 10 and 20 mg/L.

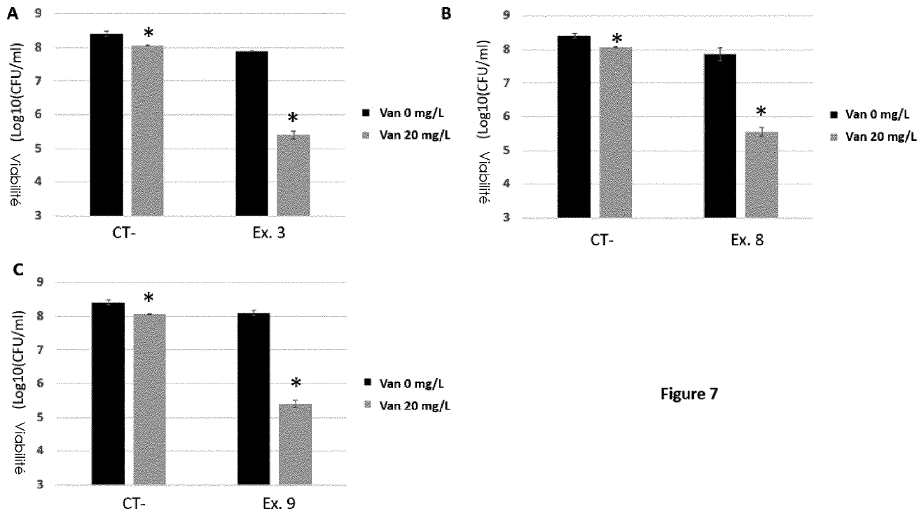

FIG. 7 shows a series of three graphs which illustrates the viability in the S. aureus ATCC33591 biofilm expressed in log₁₀ (CFU/ml) after curative treatment of the biofilm with a composition comprising a CDD enzyme cocktail comprising 500 U/ml of denarase, 7 U/ml of cellulase and 0.06 U/ml of dispersin B followed by 24 hours of incubation with 0 mg/L or 20 mg/L of vancomycin, compared with a composition comprising a BDD enzyme cocktail comprising 500 U/ml of denarase, 1% v/v of BlazePro and 0.06 U/ml of Dispersin B followed by 24 hours of incubation with 0 mg/L or 20 mg/L of vancomycin, compared with a composition comprising an FDD enzyme cocktail comprising 500 U/ml of denarase, 1% v/v Flavourzyme and 0.06 U/ml Dispersin B followed by 24 hours of incubation with 0 mg/L or 20 mg/L of vancomycin.

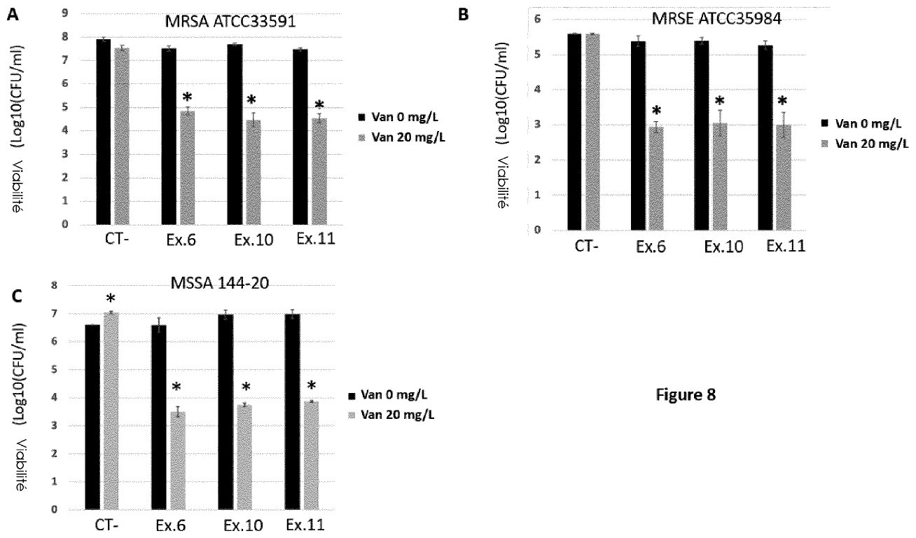

FIG. 8 shows the effect of different concentrations of vancomycin (0 and 20 mg/L) on the viability expressed in log₁₀ (CFU/ml) on the biofilm of different strains of S. aureus and S. epidermidis after curative treatment with different tri-enzyme compositions. The CDD tri-enzyme composition comprises an enzyme cocktail composed of 7 U/ml of cellulase. 0.06 U/ml of dispersin B and 500 U/ml of denarase, compared with a CDD2 enzyme composition comprising a tri-enzyme cocktail of 70 U/ml of cellulase, 0.32 U/ml of dispersin B and 500 U/ml of denarase, compared with an ADD enzyme composition comprising a tri-enzyme cocktail composed of 70 U/ml of cellulase, 2000 U/ml alpha-amylase and 500 U/ml denarase.

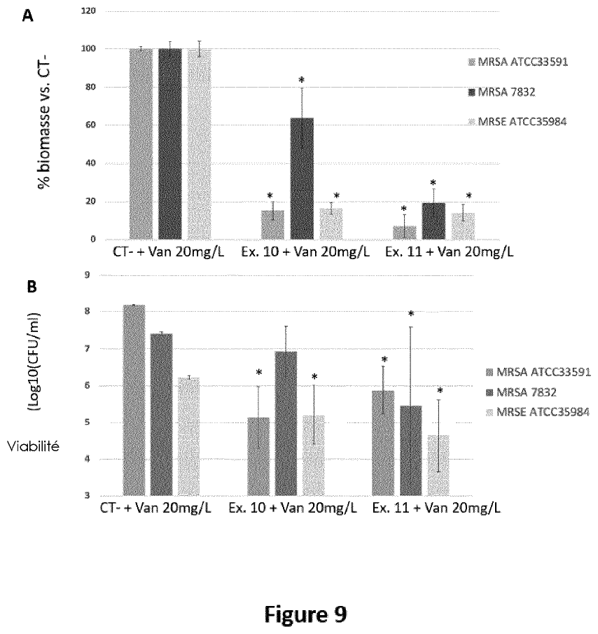

FIG. 9 shows the effect of 20 mg/L of vancomycin on the biomass expressed in % and the viability expressed in log₁₀ (CFU/ml) of the biofilm of different strains of S. aureus and S. epidermidis formed in the presence of compositions according to the invention. The CDD2 tri-enzyme composition includes a tri-enzyme cocktail composed of 70 u/ml of cellulase, 0.32 U/ml of dispersin B and 500 U/ml of denarase: compared with an ADD enzyme composition comprising a tri-enzyme cocktail consisting of 70 U/ml cellulase, 2000 U/ml alpha-amylase and 700 U/ml denarase.

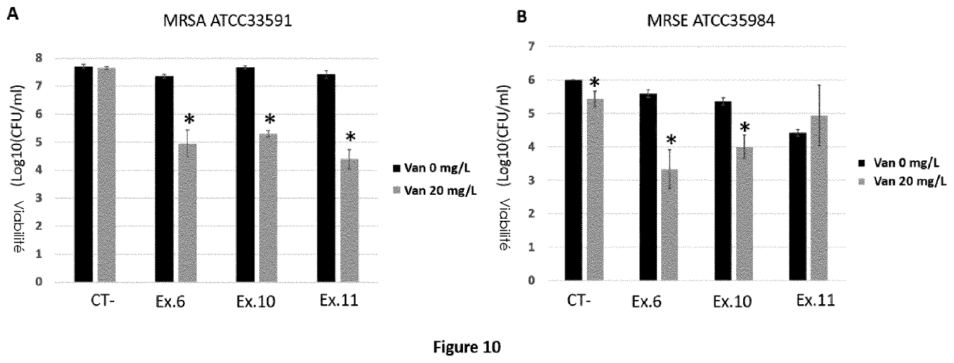

FIG. 10 shows the effect of different concentrations of vancomycin (0 and 20 mg/L) on the viability expressed in log₁₀ (CFU/ml) of the S. aureus ATCC33591 and S. epidermidis ATCC35984 biofilm formed in the presence of compositions according to invention. The CDD enzyme composition comprising a tri-enzyme cocktail composed of 7 U/ml of cellulase. 0.06 U/ml of dispersin B and 500 U/ml of denarase: The CDD2 enzyme composition comprising a ti-enzyme cocktail composed of 70 U/ml of cellulase, 0.32 U/ml of dispersin B and 500 U/ml of denarase: compared with an ADD enzyme composition comprising a tri-enzyme cocktail consisting of 70 U/ml cellulase, 2000 U/ml alpha-amylase and 500 U/ml denarase.

In the figures, identical or similar elements have the same references.

Other characteristics and advantages of the present invention will be drawn from the non-limiting description which follows, and with reference to the drawings and the examples.

### Efficacy of a Composition According to the Invention for Reducing the Biomass of Biofilms Involved in Infections of the Human Body

In order to test the efficacy of a composition according to the invention, comprising at least one endoribonuclease enzyme, several experiments were carried out on different bacterial strains (clinical isolates and ATCC collection strains) (Table 1).

TABLE 1

| Isolate/strain | species | origin |
|---|---|---|
| ATCC29213 | S. aureus MSSA | Wound (ATCC collection) |
| ATCC33591 | S. aureus MRSA | Clinical isolate (ATCC collection) |
| 7832 | S. aureus MRSA | Deep wound |
| 7841 | S. aureus MRSA | Superficial wound |
| 1142-004 | S. aureus MSSA | Wound |
| 1144-20 | S. aureus MSSA | Wound |
| ATCC35984 | S. epidermidis MRSE | Venous catheter (ATCC collection) |
| PAO1 | P. aeruginosa | Wound |
| ATCC27853 | P. aeruginosa | Infected blood (ATCC collection) |
| 618 | P. aeruginosa | Superficial wound |

The effect of a treatment with the composition according to the invention on the biomass of biofilms was studied in a static model in 96-well plates. The curative enzyme treatment was carried out on a preformed 24-hour biofilm. To test its efficacy in reducing the biomass of biofilms, the composition according to the invention was brought into contact with the biofilm for 1 hour at 37° C. The enzyme solution was then removed, and the detached biomass was removed via rinsing steps. Crystal violet (CV) dye was then used to quantify residual biomass. The latter interacts with dead and living cells, and the macromolecules of the extracellular matrix of the biofilm (such as DNA and exopolysaccharides). CV staining was quantified via spectrophotometry by measuring the absorbance at 570 nm.

### 1.1 Culture Method, Enzyme Treatment, Quantification of Biomass and Analysis of Results

The different strains of S. aureus and P. aeruginosa (Table 1) were inoculated in 5 ml of TGN culture medium (Tryptic Soy Broth VWR+1% glucose+2% NaCl) from 20 μl of a stored glycerol stock at −80° C. The strains were then aerobically incubated at 130 rpm at 37° C. After 18 hours of growth, the precultures were diluted in TGN to reach an optical density at 620 nm (OD₆₂₀) of 0.05, which corresponds to +/−5*10⁶ CFU/ml. Two hundred microlitres of each culture (n=4 for each condition tested) were placed in the wells of a 96-well plate and incubated at 37° C. without agitation for 24 hours to allow the biofilm to form. The biofilms were then washed 2× with 200 μl PBS pH 7.5 (Sigma) in order to eliminate planktonic cells (i.e. cells that do not adhere to the biofilm). The composition according to the invention or the control compositions CT− (negative control) and CT+ (positive control) (200 μl) were added to the washed biofilm and the biofilm was incubated for 1 hour at 37° C. (n=4 for each condition). The plates were then emptied, the biofilm was washed 2× with 200 μl PBS pH 7.5 (Sigma) and dried at 60° C. for 18 h before the biomass was analysed.

To quantify the residual biomass, a 1% CV solution (Sigma) was added to the biofilms at room temperature for 15 min. The unfixed dye was then washed off with distilled water. The dye attached to the biofilm was then detached and dissolved with 66% peracetic acid and absorbance at 570 nm (A570) was measured in a SpectraMax M3 multimode reader. This absorbance value corresponds to the biomass of the biofilm.

To analyse the effect of the compositions according to the invention, a statistical test (one-way ANOVA and Tukey's post-hoc test) was carried out by comparing the absorbance values of the untreated biofilms (negative control, CT−=treatment with buffer without enzyme) and biofilms treated with the enzyme compositions including a positive control, CT+. This positive control includes a dye, two non-anionic surfactants, a sequestrant, a preservative, an enzyme stabiliser, a solvent, seven enzymes including two proteases (EC 3.4), one laccase (EC 1.10.3.2), one mannanase (EC 3.2 0.1.78 and EC 3.2.1.101), one amylase (EC 3.2.1.1) and one lipase (EC 3.1.1.3). The difference is significant when the pvalue of the statistical test is less than or equal to 0.05 (* used on graphs). When this is specified in the comparative examples, the activity of the various enzyme compositions is compared (one-way ANOVA and post-hoc Tukey test).

### 1.2 Results

### Example 1.—Composition According to the Invention Containing Denarase in TGN

First, a composition comprising denarase (c-Lecta), which is an endoribonuclease from S. morcescens belonging to the EC 3.1.30 enzyme class, was tested at a concentration of 500 U/ml in TGN. The negative control (CT−) was composed of TGN only. The A₅₇₀ absorbance of the CV measured on the CT− corresponds to 100% biomass. As shown in FIG. 1, the biomass percentage of an S. aureus ATCC 29213, ATCC 33591, 7832, 7841, 1142-004 and 1144-20 biofilm decreases by 75%, 76%, 72%, 71%, 47% and 52% respectively. Statistical analysis (ANOVA and Tukey's post-hoc test) indicates that in all cases, this difference is significant (pvalue s 0.05) compared with CT−. This decrease suggests that the effect of a microbicidal agent will be potentiated after biofilm is treated with denarase.

### Comparison of the Efficacy of the Composition from Example 1 with Other Endoribonucleases

The effect on the biomass of denarase was compared to the effects of a negative control (TGN), a positive control (EnziQure® 1% in TGN: OneLife sa), and compositions containing RNase I only (Escherichia coli endoribonuclease: class EC4.6.1: ThermoFisher Scientific), and a mixture of RNase 1, RNase A (bovine endoribonuclease class EC 4.6.1: ThermoFisher Scientific) and DNase I (bovine endoribonuclease class EC 3.1.21: ThermoFisher Scientific).

The compositions are listed in Table 2.

TABLE 2

| Composition - Example | Enzyme | Concentration | Buffer |
|---|---|---|---|
| Example 1 (Ex. 1) | Denarase | 100 U/ml | TGN |
| Comparative Example 1 | RNase I | 100 U/ml | TGN |
| (CE. 1) | | | |
| Comparative Example 2 | RNase I, | 100 U/ml of | TGN |
| (CE. 2) | RNase A and | each enzyme | |
| | DNase I | | |
| Negative control CT− | (NA) | (NA) | TGN |
| Positive control CT+ | EnziQure ® | 1% | TGN |

These tests were carried out with the aim of comparing the disruptive effect on biofilms of the different nucleases at 100 U/ml in TGN. These compositions were tested on a 24-hour S. aureus ATCC33591 and 7832, and S. epidermidis ATCC35984 biofilm (n=4) by following the protocol described in point 1.1.1.

FIG. 2 shows that RNase I (endoribonuclease) according to comparative example CE1 has little or no significant effect on the decrease in the biomass percentage of the biofilms (1 biofilm/3 is significantly reduced compared to CT−). The addition of RNase A (endoribonuclease) and DNase I (endodeoxyribonuclease) in addition to RNase I according to comparative example CE2 slightly improves the disruptive activity of the composition of comparative example CE1A (2 biofilms/3 are significantly reduced). Interestingly, denarase (example 1) (endoribonuclease) significantly reduces the biomass of ⅔ biofilms, like the CE2 nuclease mixture, but more effectively: 46% vs. 30% for the ATCC33591 strain and 37% vs 13% for the ATCC35984 strain. Since the non-specific endoribonuclease activity of denarase makes it possible to digest both DNA and RNA, it is unexpected to find that an endoribonuclease specifically selected from the EC 3.1.30 or EC 3.1.31 class allows a more effective reduction of biofilm biomass than a DNase I-RNase I-RNase A mixture. The scientific reasons for this observation are not yet known.

### 1.2.3 Comparison of the Efficacy of Denarase in Curative Treatment with Other Enzyme Compositions

The anti-biomass activity in the curative treatment of denarase (500 U/ml) was then compared with that of cellulase (Sigma: 7 U/ml) and dispersin B (GIGA: 0.06 U/ml), and to that of a tri-enzyme cocktail composed of denarase, cellulase and dispersin B at the same concentrations. These four compositions (Ex1, CE3, CE4 and Ex 2) were tested on S. aureus ATCC33591, ATCC29213 and S. epidermidis ATCC35984 biofilms following the protocol detailed in point 1.1.1, with the only difference that the mixtures were made in a 20 mM Tris-HCl buffer pH 7. In this experiment, the CT− is 20 mM Tris-HCl pH 7 without enzymes and its biomass corresponds to 100%. The CT+ is composed of 1% EnziQure® in this same buffer. The compositions are listed in Table 3.

TABLE 3

| Composition - Example | Enzyme | Concentration | | Buffer |
|---|---|---|---|---|
| Example 2 (Ex. 2) | Denarase | 500 | U/ml | Tris-HCl 20 mM |
| Comparative Example 3 | Cellulase | 7 | U/ml | Tris-HCl 20 mM |
| (CE. 3) | | | | |
| Comparative Example 4 | Dispersin B | 0.06 | U/ml | Tris-HCl 20 mM |
| (CE. 4) | | | | |

| | | | |
|---|---|---|---|
| Example 3 (Ex. 3) | Denarase, | 500 U/ml | Tris-HCl 20 mM |
| | cellulase and | 7 U/ml | |
| | Dispersin B | 0.06 U/ml | |
| Negative control CT− | (NA) | (NA) | Tris-HCl 20 mM |
| Positive control CT+ | EnziQure ® | 1% | Tris-HCl 20 mM |

It can be seen in FIG. 3 that the composition comprising denarase (Ex.2) is the composition (FIG. 3A) leading to the greatest reduction in the biomass of each of the biofilms with a significant reduction and greater than 80% of the biomass of the three biofilms tested (FIG. 3A). Indeed, cellulase (CE. 3) (FIG. 3B) has no significant effect and dispersin B (CE. 4) (FIG. 3C) decreases biofilm from 70% (ATCC29213) to 27% (ATCC33591). The mixture of the 3 enzymes (Ex. 3) (FIG. 3D) is as effective as denarase alone (Ex.2) on 2 biofilms/3. However, the composition from Example 3 is less effective than the denarase from Example 2 on the biofilm of the strain ATCC29213. These results suggest that, under the experimental conditions tested, there is no synergistic effect between denarase, cellulase and dispersin B.

### 1.2.4 Comparison of the Disruptive Activity of the Biomass

To confirm the absence of synergy between denarase, cellulase and dispersin B, the disruptive activity of the biomass of denarase ([Example 2 (Ex.2)]—(FIG. 4A) was compared with that of enzyme duos denarase+dispersin B ([Example 4 (Ex.4)]—(FIG. 4B) and denarase+cellulase—([Example 5 (Ex.5)](FIG. 4C). The strains, experimental conditions and enzyme concentrations used are the same as in the experiment described above. In this experiment, the CT− is 20 mM Tris-HCl pH 7 without enzymes and its biomass corresponds to 100%. The CT+ is composed of 1% EnziQure® in this same buffer. The compositions tested are listed in Table 4.

TABLE 4

| Composition - Example | Enzyme | Concentration | Buffer |
|---|---|---|---|
| Example 2 (Ex. 2) | Denarase | 500 U/ml | Tris-HCl 20 mM |
| Example 4 (Ex. 4) | Denarase and | 500 U/ml and | Tris-HCl 20 mM |
| | Dispersin B | 0.06 U/ml | |
| Example 5 (Ex. 5) | Denarase and | 500 U/ml and | Tris-HCl 20 mM |
| | cellulase | 7 U/ml | |
| Negative control CT− | (NA) | (NA) | Tris-HCl 20 mM |
| Positive control CT+ | EnziQure ® | 1% | Tris-HCl 20 mM |

Similarly to what was observed previously, the results obtained indicate that the addition of dispersin B (Example 4—FIG. 4B) or cellulase (Example 3—FIG. 4C) does not significantly increase the activity of denarase from Example 2 on the strains of staphylococci tested (FIG. 4A). We therefore do not observe any synergistic effect associated with the combination of denarase with cellulase or dispersin B.

### 1.2.5 Comparison of Disruptive Activity on P. aeruginosa

The disruptive activity on the biofilm of denarase according to Example 1 (500 U/ml) and of a tri-enzymatic mixture according to Example 6 composed of denarase (500 U/ml), cellulase (7 U/ml) and dispersin B (0.06 U/ml) was tested on three strains of P. aeruginosa (PAO1, ATCC27853 and 618) as a curative treatment (TGN buffer). In this experiment, the CT− is the TGN without enzymes and its biomass corresponds to 100%. The CT+ is composed of 1% EnziQure® in this same buffer.

The compositions tested are listed in Table 5.

TABLE 5

| Composition - Example | Enzyme | Concentration | Buffer |
|---|---|---|---|
| Example 1 (Ex. 1) | Denarase | 500 U/ml | TGN |
| Example 6 (Ex. 6) | Denarase and | 500 U/ml and | TGN |
| | Dispersin B | 0.06 U/ml | |
| | and cellulase | 7 U/ml | |
| Negative control CT− | (NA) | (NA) | TGN |
| Positive control CT+ | EnziQure ® | 1% | TGN |

The results presented in FIG. 5 show that denarase (Ex.1: FIG. 5B) and the tri-enzymatic composition from Example 6 (Ex. 6: FIG. 5A) are equally effective in reducing the biomass of the three biofilms tested with the respective reductions of 94%, 85%, and 44% in the biomass of P. aeruginosa PAO1, ATCC27853, and 618 biofilms, in the presence of denarase. No significant difference is observed between the two compositions. These results indicate that there is no synergy between denarase, cellulase and dispersin B to reduce the biomass of P. aeruginosa biofilms. Also, interestingly, the denarase and composition from Example 6 is more effective than the multi-enzyme detergent enziQure (CT+: contains seven enzymes) in decreasing the biomass of the P. aeruginosa 618 biofilm.

### Efficacy of a Composition According to the Invention in Combination with a Microbicide to Reduce the Biomass and the Viability of Biofilms Involved in Infections of the Human Body

For the tests described below, vancomycin was used as the antibiotic molecule. The antibiotic was added at different concentrations in TGN for 24 hours at 37° C. on the biofilms pretreated with the enzyme composition according to the invention (see the below sections). The biofilm was then washed before carrying out the biomass and viability measurements.

Two different biofilm models were used: static biofilm in 96-well plate (as described previously) and biofilm on titanium coupons. Titanium was chosen because it is a material widely used in the manufacture of implants (e.g. orthopaedic prostheses, dental implants, heart valves, pacemakers, etc.). The techniques used to measure viability differ between the two models and are detailed below.

### 2.1 Biofilm in 96-Well Plates

In this model, cell viability was measured indirectly, by measuring the metabolism of cells in the biofilm. Indeed, viability was quantified by tracking changes in the colour of resazurin (7-Hydroxy-3H-phenoxazin-3-one 10 oxide) (prestoBlue, ThermoFisher). This blue coloured compound turns pink when reduced to resorufin by the redox activity of the biofilm, activity which is directly proportional to the number of metabolically active cells in the biofilm. The quantification of resorufin was carried out by measuring fluorescence. To correlate the number of cells present in the biofilm with the fluorescent signal, a calibration curve was produced in each experiment by measuring the fluorescent signal emitted by various known cell concentrations.

### 2.1.1 Method of Culture, of Enzyme Treatment, of Quantifying Biomass and Viability and Analysing the Results

The experimental method for cultivating the strains and forming the biofilm is described in point 1.1 of this document. After 24 hours of biofilm formation at 37° C. in the wells of the 96-well plate, the biofilm was washed 2× with PBS (200 μl) and treated for 1 hour at 37° C. with the composition according to the invention or the control compositions (CT− and CT+) (n=4 for each condition tested). The biofilm was then washed 2× with PBS (200 μl). Two hundred microlitres of different concentrations of vancomycin prepared in TGN were then added to the biofilm and the plate was incubated for 24 hours at 37° C. Biofilms that were not treated with vancomycin were incubated in the presence of TGN only. The biofilms were then washed 2× with PBS (200 μl) before biomass and viability were analysed. Biomass was quantified via the CV staining technique following the procedure described in point 1.1. For the analysis of biomass, prestoBlue reagent (ThermoFisher) was diluted at a ratio of 1:10 in the CA-MHB (VWR) culture medium and 200 μl were added to the wells. To calculate the calibration curve, the biofilm of two CT− samples not treated with enzymes or with vancomycin was detached and homogenised in 100 μl of CA-MHB and different dilutions of this sample were carried out in CA-MHB (from 10 to 10). Ten microlitres of each dilution were then spread out on a TSA dish to perform a bacterial count. One hundred microlitres of a diluted 5×-CaMHB prestoBlue mixture were then added to 90 μl of the different dilutions. The plate containing all the samples was then incubated for 24 hours at 37° C. in the spectraMax M3 plate reader. Fluorescence was measured every 10 minutes for 18 hours (excitation 560 nm, emission 590 nm).

To analyse the effect of the compositions according to the invention, a statistical test (one-way ANOVA and Tukey's post-hoc test) was carried out. This test compares the values of fluorescence (viability) or absorbance at 570 nm (biomass) of the negative control CT− (=treatment with buffer without enzymes) and of biofilms treated with the enzyme compositions, and does so at each vancomycin concentration. The difference is significant when the pvalue of the statistical test is less than or equal to 0.05 (* used on graphs). When this is specified in the comparative examples, the activity of the various enzyme compositions is compared (one-way ANOVA and post-hoc Tukey test).

### 2.1.2 Results

FIG. 6 illustrates the potentiated effect of a microbicidal agent (vancomycin) as a result of the composition from Example 1 or Example 2 comprising denarase on the S. aureus ATCC33591 biofilm. The composition was tested in TGN (Ex. 1—FIGS. 6A and 6C) and in 20 mM TrisHCl pH7 (Ex. 2—FIGS. 6B and 6D). The CT− and CT+ controls were composed of buffer without enzymes. The CT+ controls were composed of 1% enziQure® (v/v) in buffer (TGN or 20 mM TrisHCl). After enzyme treatment for 1 hour at 37° C., the biofilms were incubated for 24 hours at 37° C. in the presence of the following concentrations of vancomycin: 0 mg/L, 10 and 20 mg/L.

The viability results obtained (FIGS. 6A and 6B) show that in the absence of enzyme treatment, vancomycin (10 mg and 20 mg/L) does not reduce (FIG. 5A) or only very slightly reduces (FIG. 5B) the viability of the S. aureus ATCC33591 biofilm. On the other hand, when the biofilm is pretreated with denarase from Example 1 (in TGN) and Example 2 (in 20 mM TrisHCl pH7), a significant reduction compared to the CT− of 1.35 logs and 0.5 logs, respectively, is observed after treatment with 20 mg/L of vancomycin. These decreases correspond to a decrease in viability of 96.5 and 80%, respectively, compared to the respective CT− controls without enzymes at 20 mg/L of vancomycin. The potentiating effect of denarase on vancomycin 20 mg/L is also observed on biomass with a reduction in biomass of 84% and 70% in TGN and Tris, respectively (FIGS. 6C and 6D). A similar but less significant effect on viability and biomass was measured in the presence of 10 mg/L vancomycin: 50% reduction in viability and 80% reduction in biomass in TGN, compared to CT−.

### 2.2 Biofilm on Titanium Coupons

In this model, viability was quantified after detachment and homogenisation of the biofilm via the technique of spreading and counting cells on a TSA culture media (VWR).

### 2.2 Method of Culture, of Enzyme Treatment, of Quantifying Biomass and Viability and Analysing the Results

Titanium coupons (n=3 for each condition) were placed in the wells of a 12-well plate and incubated at 37° C. with agitation (50 rpm) for 24 hours in 3 ml of bacterial inoculum at an OD of 0.005 (106 CFU/ml) in order to allow biofilm to form. The coupons were then washed 2× with PBS (2 ml) and treated for 1 hour at 37° C. with the composition according to the invention or the solution without enzymes (CT−=20 mM TrisHCl pH7). The coupons were then washed twice with PBS (2 ml) and reincubated in 750 μl of TGN containing 0 or 20 mg/L of vancomycin for 24 hours at 37° C. with agitation (50 rpm). After two washes with PBS (2 ml), the biofilm was detached and homogenised in 2 ml of PBS. Different dilutions were carried out (from 10 to 10) and 10 μl of each dilution was spread on a TSA dish. Colonies (CFU) were counted after 18 hours of incubation at 37° C.

To analyse the effect of the compositions according to the invention, a statistical test (one-way ANOVA and Tukey's post-hoc test) was carried out. This test compares the fluorescence (viability) or absorbance (biomass) values of the CT− negative control (=treatment with buffer without enzymes) and of the biofilms treated with the enzyme compositions, at each vancomycin concentration. The difference is significant when the pvalue of the statistical test is less than or equal to 0.05 (* used on graphs). When this is specified in the comparative examples, the activity of the various enzyme compositions is compared (one-way ANOVA and post-hoc Tukey test).

### 2.1.2 Results

### Potentiating Effect of Three Ti-Enzymatic Compostions on the Efficacy of Vancomycin

FIG. 7 illustrates the potentiating effect of three tri-enzyme compositions containing denarase on the microbicidal effect of vancomycin at 20 mg/L: (i) Example 8 (Ex. 8): cellulase (7 U/ml), denarase (500 U/ml) and dispersin B (0.06 U/ml), (ii) Example 9 (Ex. 9): flavourzyme (1% v/v), denarase (500 U/ml) and dispersin B (0.06 U/ml) and (iii) BDD: Blaze® Pro (1% v/v), denarase (500 U/ml) and dispersin B (0.06 U/ml) on S. aureus ATCC33591. The buffer used is 20 mM Tris-HCl pH7.

The compositions are listed in Table 6.

TABLE 6

| Composition - Example | Enzyme | Concentration | Buffer |
|---|---|---|---|
| Example 3 (Ex. 3) | Denarase and | 500 U/ml and | Tris-HCl 20 mM |
| | Dispersin B and | 0.06 U/ml | |
| | cellulase | 7 U/ml | |
| Example 8 (Ex. 8) | Denarase and | 500 U/ml | Tris-HCl 20 mM |
| | Flavourzyme | 1% v/v | |
| | And Dispersin B | 0.06 U/ml | |
| Example 9 (Ex. 9) | Denarase and | 500 U/ml | Tris-HCl 20 mM |
| | Blaze ® Pro | 1% v/v | |
| | And Dispersin B | 0.06 U/ml | |
| Negative control CT− | (NA) | (NA) | Tris-HCl 20 mM |
| Positive control CT+ | EnziQure ® | 1% | Tris-HCl 20 mM |

The results show that in the absence of enzyme treatment (CT−) vancomycin 20 mg/L does not decrease viability in biofilm. On the other hand, the pretreatment of biofilms with the three compositions significantly potentiate the effect of vancomycin. Indeed, with the three cocktails a similar reduction of 2.5 logs is observed in the presence of 20 mg/L of vancomycin (=99.8% reduction in viability).

### Potentiating Effect of 3 Tri-Enzymatic Compostions on Vancomycin

FIG. 8 illustrates the potentiating effect of three tri-enzyme compositions containing denarase on the microbicidal effect of vancomycin at 20 mg/L: (i) Example 6: cellulase (7 U/ml), denarase (500 U/ml) and dispersin B (0.06 U/ml), (ii) Example 10: cellulase (70 U/ml), denarase (500 U/ml) and dispersin B (0.32 U/ml) and (iii) Example 11: alpha-amylase (2000 U/ml): cellulase (70 U/ml), denarase (500 U/ml) and dispersin B (0.32 U/ml). These cocktails were tested as a curative treatment on S. aureus ATCC33591 (FIG. 8A), S. epidermidis ATCC35984 (FIG. 8B) and S. aureus 144-20 (FIG. 8C) biofilms. The buffer used is TGN.

The compositions are listed in Table 7.

TABLE 7

| Composition - Example | Enzyme | Concentration | Buffer |
|---|---|---|---|
| Example 6 (Ex. 6) | Denarase and | 500 U/ml and | TGN |
| | Dispersin B and | 0.06 U/ml | |
| | cellulase | 7 U/ml | |
| Example 10 (Ex. 10) | Denarase and | 500 U/ml | TGN |
| | Dispersin B and | 0.32 U/ml | |
| | cellulase | 70 U/ml | |
| Example 11 (Ex. 11) | Denarase and | 500 U/ml | TGN |
| | alpha-amylase | 2000 U/ml | |
| | And Dispersin B | 0.32 U/ml | |
| Negative control CT− | (NA) | (NA) | TGN |
| Positive control CT+ | EnziQure ® | 1% | TGN |

The results show that in the absence of enzyme treatment (CT−) vancomycin 20 mg/L does not significantly reduce viability in biofilm. On the other hand, the pretreatment of biofilms with the three compositions significantly potentiates the effect of vancomycin on the viability of the three strains tested. Under these conditions, a reduction in viability of 2.5 to 3 logs was observed in the presence of 20 mg/L vancomycin compared with the CT−'s not treated with the cocktails.

### Efficacy of a Composition According to the Invention in Combination with a Microbicide to Prevent the Formation of Biofilm Involved in Infections of the Human Body

### 3.1 Model of Biofilm in 96-Well Plates

The capacity of the composition according to the invention to prevent the formation of biofilm was tested on a model of biofilm in a 96-well plate. In this experimental set-up, the enzyme compositions were added to the initial bacterial inoculum (TGN medium containing 5*106 CFU/ml), i.e. before biofilm formation. After 24 hours of growth at 37° C., the biofilms were washed and incubated in the presence of vancomycin (20 mg/L) for 24 hours at 37° C., as described in point 2.1.1. The biomass value (CV staining) and the viability (measurement of metabolism) of the biofilms were then measured and compared with the CT− condition where the starting inoculum did not contain enzymes (n=4 in each condition).

The compositions tested are listed in Table 8.

TABLE 8

| Composition - Example | Enzyme | Concentration | Buffer |
|---|---|---|---|
| Example 10 (Ex. 10) | Denarase and | 500 U/ml | TGN |
| | Dispersin B and | 0.32 U/ml | |
| | cellulase | 70 U/ml | |
| Example 11 (Ex. 11) | Denarase and | 500 U/ml | TGN |
| | alpha-amylase | 2000 U/ml | |
| | And Dispersin B | 0.32 U/ml | |
| Negative control CT− | (NA) | (NA) | TGN |
| Positive control CT+ | EnziQure ® | 1% | TGN |

FIG. 9 shows the preventive effect of two compositions according to the invention containing denarase (examples 10 & 11), on different strains of S. aureus and S. epidermidis. In the presence of 20 mg/L vancomycin, the two cocktails significantly reduce the quantity of biomass formed (FIG. 9A) and the viability of the biofilm (FIG. 9B), in varying degrees between the strains.

### 3.2 Titanium Coupon Biofilm Model

The preventive effect on biofilm formation of the compositions according to the invention was tested using a titanium coupon model. In this experiment, the coupons were incubated for 24 hours at 37° C. with a bacterial inoculum (106 CFU/ml) in TGN containing different enzyme compositions, or not (=CT−). After biofilm formation, coupons were incubated in the presence (Van Omg/L) or the presence of 20 mg/L of vancomycin for 24 hours before analysing viability using the technique of spreading on TSA and counting.

The compositions tested are listed in Table 7.

TABLE 7

| Composition - Example | Enzyme | Concentration | Buffer |
|---|---|---|---|
| Example 6 (Ex. 6) | Denarase and | 500 U/ml and | TGN |
| | Dispersin B and | 0.06 U/ml | |
| | cellulase | 7 U/ml | |
| Example 10 (Ex. 10) | Denarase and | 500 U/ml | TGN |
| | Dispersin B and | 0.32 U/ml | |
| | cellulase | 70 U/ml | |
| Example 11 (Ex. 11) | Denarase and | 500 U/ml | TGN |
| | alpha-amylase | 2000 U/ml | |
| | And Dispersin B | 0.32 U/ml | |
| Negative control CT− | (NA) | (NA) | TGN |
| Positive control CT+ | EnziQure ® | 1% | TGN |

FIG. 10 illustrates the preventive effect of three tri-enzyme compositions containing denarase on the microbicidal effect of vancomycin at 20 mg/L: (i) Example 6: cellulase (7 U/ml), denarase (500 U/ml) and dispersin B (0.06 U/ml), (ii) Example 10: cellulase (70 U/ml), denarase (500 U/ml) and dispersin B (0.32 U/ml) and (iii) Example 11: alpha-amylase (2000 U/ml): cellulase (70 U/ml), denarase (500 U/ml) and dispersin B (0.32 U/ml). These cocktails were tested as preventive treatment on the S. aureus ATCC33591 biofilm (FIG. 10A) and S. epidermidis ATCC35984 biofilm (FIG. 10B).

The results indicate that in the presence of vancomycin 20 mg/L the three cocktails significantly reduce viability in the biofilm formed by S. aureus ATCC33591 (decrease of 2.5 to 3 logs) (FIG. 10A) and S. epidermidis ATCC35984 (decrease of 2 to 3 logs) (FIG. 10B) after 24 hours of incubation. These observations show that the compositions according to the invention containing denarase prevent biofilm formation, and consequently potentiate the effect of an antibiotic on its capacity to reduce viability within biofilm.

It is understood that the present invention is in no way limited to the embodiments described above and that many modifications can be made thereto without departing from the scope of the attached claims.

For example, denarase was used in the examples, but it goes without saying that the examples could have illustrated the activity of the Benzonase® Endonuclease provided by the company Millipore Sigma

### 4.1 Biofilms Comprising Candida

The inventors then attempted to treat biofilms comprising Candida albicans, either in monoculture or in complex biofilm models comprising Candida albicans and prokaryotes. Different strains of Candida albicans were used: the ATCC24433 strain, as well as strains isolated from orthopaedic prosthesis infections. Subtilisin (protease from Bacillus licheniformis) and lyticase (from Arthrobacter luteus or Bacillus subtilis) were obtained at Sigma. The other enzymes are those used above.

The antifungal drug Caspofungin (Merck Sharp & Dohme) and, for complex biofilms additionally comprising one or more prokaryotes, the antibiotics Moxifloxacin HCl (Bayer) or Meropenem (Mylan) were used (24 hours of treatment, after the application of enzymes).

Cytotoxicity tests were performed on MG63 osteoblasts and other cell types.

Both subtilisin (already at concentrations of about 0.5 U/mL) and Lyticase (already at 10 U/mL) are toxic to osteoblasts, but not necessarily toxic to other cells. These are the concentrations that will be used on biofilms.

Surprisingly, the inventors observed that subtilisin (two treatment durations were tested: 30 minutes and 1 hour) sensitised biofilms comprising Candida albicans (associations with Gram-positive bacteria, here, S. aureus and/or Gram-negative bacteria: here. E. coli, were tested), or consisting solely of Candida albicans. A combination of Denarase® with Lyticase (combination applied for 30 minutes or 1 hour) also showed a synergistic effect on these biofilms. The combination of subtilisin with Denarase® did not show any advantage over the effect obtained by subtilisin alone on biofilms comprising Candida albicans.

The inventors tested a sequential application of a composition comprising Denarase, then subtilisin, with increased efficacy.

1. A method of treatment with a parapharmaceutical or pharmaceutical composition, the parapharmaceutical or pharmaceutical composition comprising at least one endoribonuclease enzyme selected from the group consisting of enzymes belonging to the enzyme classes EC 3.1.30 and EC 3.1.31 and mixtures thereof, to potentiate a microbicidal agent, comprising an antibiotic, an antifungal, or a disinfectant, the method comprising administering a therapeutically effective dose of the parapharmaceutical or pharmaceutical composition to human beings in the treatment and/or the prevention of bacterial infections involving biofilm formation.

2. The method of treatment with a parapharmaceutical or pharmaceutical composition according to claim 1, wherein the treatment and/or prevention of bacterial infections involving biofilm formation is a curative or preventive treatment for dermatological infections or infections developing on burns and superficial or deep wounds.

3. The method of treatment with a parapharmaceutical or pharmaceutical composition according to claim 1, wherein the treatment and/or prevention of bacterial infections involving biofilm formation is a curative and/or preventive treatment for post-implantation infections associated with infection of tissues around a medical device implanted in the body or of a medical device implanted in the body.

4. The method of treatment with a parapharmaceutical or pharmaceutical composition according to claim 1, in a personal hygiene or cosmetic treatment, comprising nail care, an oral solution, a mouthwash, a toothpaste, an eye bath, an eye lens cleaning solution, a cleaning solution for dental appliances/prostheses, toothbrushes, skin care for acne.

5. The method of treatment with a parapharmaceutical or pharmaceutical composition according to claim 1, wherein said composition further comprises a series of additional enzymes comprising one, two, three, four, five, six, seven, or eight enzyme(s).

6. The method of treatment with a parapharmaceutical or pharmaceutical composition according to claim 5, wherein an enzyme from said series of additional enzymes of the said composition is selected from the group consisting of glycosidases (EC 3.2.1), deoxyribonucleases (EC 3.1.21), oxidoreductases (EC 1.), carboxylic ester hydrolases (EC 3.1.1), proteases and peptidases (EC 3.4.), and mixtures thereof.

7. The method of treatment with a parapharmaceutical or pharmaceutical composition according to claim 1, wherein said composition comprises at least a second enzyme selected from the group of glycosidases (EC 3.2.1), preferably a glucanase as comprising endo-β-1 3-glucanase, and/or an endoglucanase from the group EC.3.2.1.39.

8. The method of treatment with a parapharmaceutical or pharmaceutical composition according to claim 7, wherein said composition comprises at least one third enzyme selected from the group consisting of glycosidases (EC 3.2.1), deoxyribonucleases (EC 3.1.21), oxidoreductases (EC 1), carboxylic ester hydrolases (EC 3.1.1), peptidases and proteases (EC 3.4), and mixtures thereof, selected from the group consisting of glycosidases (EC 3.2.1) and peptidases (EC 3.4), the composition comprises at least dispersin B, cellulase and endoglucanase (β 1,3).

9. The method of treatment with a parapharmaceutical or pharmaceutical composition according to claim 1, wherein the treatment and/or prevention of bacterial Infections involving biofilm formation is a curative or preventive treatment for a bacterial infection of tissues surrounding an implanted orthopaedic implant or of the implanted orthopaedic implant.

10. The method of treatment with a parapharmaceutical or pharmaceutical composition according to claim 1, wherein the treatment and/or prevention of bacterial infections involving biofilm formation is a curative or preventive treatment for a bacterial infection of tissues surrounding an implanted dental implant or of the implanted dental implant.

11. The method of treatment with a parapharmaceutical or pharmaceutical composition according to claim 1, wherein the treatment and/or prevention of bacterial infections involving biofilm formation is a curative or preventive treatment for a bacterial infection of tissues surrounding a catheter, a cannula, a probe, a prosthesis, an endosseous implant, a zygomatic implant, an orthodontic implant, a dental prosthesis, a retainer, a valve, a drain, a stent, a tube for artificial respiration or an implanted screw or of a catheter, a cannula, a probe, a prosthesis, an endosseous implant, a zygomatic implant, an orthodontic implant, a dental prosthesis, a retainer, a valve, a drain, a stent, an artificial respiration tube or an implanted screw.

12. The method of treatment with a parapharmaceutical or pharmaceutical composition according to claim 1, wherein the treatment and/or prevention of bacterial infections involving biofilm formation comprises an application of said composition to a wound, an infection, tissues or a medical device, comprising an application of a woven or non-woven support impregnated with said parapharmaceutical or pharmaceutical composition.

13. The method of treatment with a parapharmaceutical or pharmaceutical composition according to claim 1, wherein the treatment and/or prevention of bacterial infections involving biofilm formation comprises an application of said composition to a wound, an infection, tissues, comprising an application of a dressing in the form of a gel comprising said parapharmaceutical or pharmaceutical composition.

14. The method of treatment with a parapharmaceutical or pharmaceutical composition according to claim 1, wherein the treatment and/or prevention of bacterial infections involving biofilm formation comprises an application of said composition to a wound, infection, tissues or a medical device, comprising an application of an aqueous, buffered solution of said parapharmaceutical or pharmaceutical composition, by infiltration, by irrigation, by injection, by percutaneous application, by inhalation, by mouthwash or eyewash, or by dabbing.

15. The method of treatment with a parapharmaceutical or pharmaceutical composition according to claim 1, wherein the treatment and/or prevention of bacterial infections involving biofilm formation comprises an application of said composition to a wound, infection, tissue or medical device, by an application of a paste, cream, ointment, or a viscous solution comprising said parapharmaceutical or pharmaceutical composition.

16. The method of treatment with a parapharmaceutical or pharmaceutical composition according to claim 1, wherein said said antibiotic and/or antifungal, phage, or said disinfectant and said composition form a combination product for simultaneous, separate or staggered use over time.

17. Parapharmaceutical or pharmaceutical composition for administration to human beings, comprising at least one endoribonuclease enzyme selected from classes EC 3.1.30 and EC 3.1.31, at a concentration of 10 to 1000 U/ml.

18. The parapharmaceutical or pharmaceutical composition according to claim 17, wherein said at least one endoribonuclease enzyme is of bacterial origin and/or belongs to class EC 3.1.30.1 or EC 3.1.30.2.

19. The parapharmaceutical or pharmaceutical composition according to claim 17, further comprising a series of additional enzymes, comprising one, two, three, four, five, six, seven, or eight enzyme(s).

20. The parapharmaceutical or pharmaceutical composition according to claim 19, wherein an enzyme from said series of additional enzymes of said composition is selected from the group consisting of glycosidases (EC 3.2.1), deoxyribonucleases (EC 3.1.21), oxidoreductases (EC 1), carboxylic ester hydrolases (EC3.1.1) and peptidases (EC 3.4), and mixtures thereof, the additional enzyme is glucanase comprising endo-β-1 3-glucanase, and/or an endoglucanase from the group EC.3.2.1.39.

21. The parapharmaceutical or pharmaceutical composition according to claim 17, further comprising at least a second enzyme selected from the group of glycosidases (EC 3.2.1).

22. The parapharmaceutical or pharmaceutical composition according to claim 21, further comprising at least a third enzyme selected from the group comprising glycosidases (EC 3.2.1), deoxyribonucleases (EC 3.1.21), oxidoreductases (EC 1), carboxylic ester hydrolases (EC 3.1.1), proteases and peptidases (EC 3.4).

23. The parapharmaceutical or pharmaceutical composition according to claim 22, comprising at least a fourth enzyme selected from the group comprising glycosidases EC 3.2.1), deoxyribonucleases (EC 3.1.21), oxidoreductases (EC 1), carboxylic ester hydrolases (EC 3.1.1), proteases and peptidases (EC 3.4), comprising as additional enzymes a glucanase comprising endo-β-1 3-glucanase and/or an endoglucanase from the group EC.3.2.1.39, Dispersin B and cellulase.

24. The parapharmaceutical or pharmaceutical composition according to claim 23, comprising at least a fifth enzyme selected from the group comprising glycosidases (EC 3.2.1), deoxyribonucleases (EC 3.1.21), oxidoreductases (EC 1), carboxylic ester hydrolases (EC 3.1.1), proteases and peptidases (EC 3.4).

25. The parapharmaceutical or pharmaceutical composition according to claim 24, comprising at least a sixth enzyme selected from the group comprising glycosidases (EC 3.2.1), deoxyribonucleases (EC 3.1.21), oxidoreductases (EC 1), carboxylic ester hydrolases (EC 3.1.1), proteases and peptidases (EC 3.4).

26. The parapharmaceutical or pharmaceutical composition according to claim 25, comprising at least a seventh enzyme selected from the group comprising glycosidases (EC 3.2.1), deoxyribonucleases (EC 3.1.21), oxidoreductases (EC 1), carboxylic ester hydrolases (EC 3.1.1), proteases and peptidases (EC 3.4).

27. The parapharmaceutical or pharmaceutical composition according to claim 26, comprising at least an eighth enzyme selected from the group comprising glycosidases (class EC 3.2.1), deoxyribonucleases (class EC 3.1.21), oxidoreductases (EC class 1.), carboxylic ester hydrolases (EC class 3.1.1), proteases and peptidases (EC class 3.4.), and mixtures thereof.

28. The parapharmaceutical or pharmaceutical composition according to claim 17, further comprising at least one microbicidal molecule comprising an antibiotic, an antiseptic, antifungal, a phage, or one or more microbicidal peptides conditioned separately or together with said endoribonuclease.

29. The parapharmaceutical or pharmaceutical composition according to claim 17, further comprising a quorum sensing inhibitor.

30. The parapharmaceutical or pharmaceutical composition according to claim 17, in the form of a solution, further comprising an enzyme buffer selected from the group consisting of Tris-HCl, TGN, TBS, PBS, HEPES, MES, PIPES, MOPS, BES, TES, phosphate buffer and citrate buffer, containing 0 to 2 mM MgCl2, containing 0 to 2 mM CaCl2), and 0 to 500 mM NaCl.

31. The parapharmaceutical or pharmaceutical composition to claim 17, in the form of a solution, further comprising an enzyme buffer comprising 0 to 50% of stabilising agent comprising polyol, arginine, calcium formate, or glucose.

32. The parapharmaceutical or pharmaceutical composition according to claim 17, further comprising at least one surfactant.

33. The parapharmaceutical or pharmaceutical composition according to claim 17, further comprising at least one preservative.

34. The parapharmaceutical or pharmaceutical composition according to claim 17, further comprising at least one sequestrant.

35. The parapharmaceutical or pharmaceutical composition according to claim 17, in the form of a solution, for example a mouthwash, an eye bath, a lotion, an irrigation solution, a solution for injection.

36. The parapharmaceutical or pharmaceutical composition according to claim 17, in the form of a hydrophilic dressing, for example a hydrogel.

37. The parapharmaceutical or pharmaceutical composition according to claim 17, in immobilised form on a woven or non-woven, dry support or on a medical device or in impregnated form on a woven or non-woven support.

38. The parapharmaceutical or pharmaceutical composition according to claim 17, in the form of a topical composition.

39. The parapharmaceutical or pharmaceutical composition according to claim 17, in the form of a sterile solution which for administration by infiltration, by irrigation, by injection, by percutaneous application and by inhalation.

40. The parapharmaceutical or pharmaceutical composition according to claim 17, adapted as a potentiator for a microbicidal agent, comprising an antibiotic and/or an antifungal, a phage, or a disinfectant, in the treatment and/or the prevention of bacterial infections involving biofilm formation.

41. The parapharmaceutical or pharmaceutical composition according to claim 17, for a curative or preventive treatment for dermatological infections or infections developing on burns and superficial or deep wounds.

42. The parapharmaceutical or pharmaceutical composition according to any one of claims 17 to 38 claim 17, for curative and/or preventive treatment of post-implantation infections associated with the infection of tissues around a medical device implanted in the body or of a medical device implanted in the body.

43. The parapharmaceutical or pharmaceutical composition according to claim 17, for a personal hygiene or cosmetic treatment, comprising nail care, an oral solution, a mouthwash, a toothpaste, eye bath, eye lens cleaning solution, dental appliance cleaning solution, toothbrush cleaning solution, skin care for acne.

44. The parapharmaceutical or pharmaceutical composition according to claim 17, for a curative or preventive treatment for a bacterial infection of tissues surrounding an implanted orthopaedic implant or of the implanted orthopaedic implant.

45. The parapharmaceutical or pharmaceutical composition according to claim 17, for a curative or preventive treatment for a bacterial infection of tissues surrounding an implanted dental implant or of the implanted dental implant.

46. The parapharmaceutical or pharmaceutical composition according to claim 17, for a curative or preventive treatment of a bacterial infection of tissues surrounding a catheter, a cannula, a probe, a prosthesis, an endosseous implant, a zygomatic implant, an orthodontic implant, a dental prosthesis, a retainer, a valve, a drain, a stent, a tube for artificial respiration or an implanted screw or of a catheter, a cannula, a probe, a prosthesis, an endosseous implant, a zygomatic implant, an orthodontic implant, a dental prosthesis, a retainer, a valve, a drain, a stent, a tube for artificial respiration or an implanted screw.

47. The parapharmaceutical or pharmaceutical composition according to claim 17, for treatment and/or prevention of bacterial infections involving biofilm formation which comprises an application of said composition to a wound, infection, tissues or a medical device, by an application of a woven or non-woven support impregnated with said parapharmaceutical or pharmaceutical composition or on which said parapharmaceutical or pharmaceutical composition is immobilised.

48. The parapharmaceutical or pharmaceutical composition according to claim 17, for treatment and/or prevention of bacterial infections involving biofilm formation which comprises an application of said composition to a wound, an infection, tissues, by an application of a dressing in gel form, comprising a hydrogel comprising said parapharmaceutical or pharmaceutical composition.

49. The parapharmaceutical or pharmaceutical composition according to claim 17, for treatment and/or prevention of bacterial infections involving biofilm formation which comprises an application of said composition to a wound, infection, tissues or a medical device, by an application of an aqueous buffered solution of said parapharmaceutical or pharmaceutical composition, by infiltration, by irrigation, by injection, by percutaneous application, by inhalation, by mouthwash or eyewash, by dabbing, or by soaking.

50. The parapharmaceutical or pharmaceutical composition according to claim 17, for treatment and/or prevention of bacterial infections involving biofilm formation which comprises an application of said composition to a wound, an infection, tissues or a medical device, by an application of a paste or of a viscous solution comprising said parapharmaceutical or pharmaceutical composition.

51. The parapharmaceutical or pharmaceutical composition according to claim 28, for a combination product for simultaneous, separate or staggered use over time.

52. The parapharmaceutical or pharmaceutical composition according to claim 17, for surgical use.

53. A non-therapeutic method of using subtilisin (EC.3.4.21.62) comprising potentiating one or more anti-microbiological molecules for a biofilm comprising Candida albicans.

54. The method according to claim 53, wherein the anti-microbiological molecules are an antifungal and/or an antibiotic, wherein said antibiotic is an antibiotic effective against Gram-negative bacteria in planktonic form and Gram-positive bacteria in planktonic form.

55. Pharmaceutical composition comprising subtilisin (EC.3.4.21.62) for the treatment of a biofilm comprising Candida albicans, said biofilm affecting a patient.

56. The pharmaceutical composition according to claim 55 for sequential use with an antifungal agent and/or an antibiotic, preferably wherein the antifungal is active against Candida albicans (in planktonic form) and the antibiotic is active against Gram-negative and/or Gram-positive bacteria (in planktonic forms).

57. The pharmaceutical composition according to claim 55 for curative treatment of tissues surrounding an orthopaedic implant, a catheter, a cannula, a probe, a prosthesis, an endosseous implant, a zygomatic implant, an orthodontic implant, a dental prosthesis, a retainer plate, a valve, a drain, a stent, a tube for artificial respiration or an implanted screw, of an epidermis, a wound or a mucosa inside the body being infected with a biofilm comprising Candida albicans.
